SEQUENCE LISTING (18 sequences)

SEQ ID NO: 1 - protein, 143 aa, synthetic construct
  REGION 1..143: note = Recombinant MYDGF variant +A
AVSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKASRTEL

SEQ ID NO: 2 - protein, 143 aa, synthetic construct
  REGION 1..143: note = Recombinant MYDGF variant +S
SVSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKASRTEL

SEQ ID NO: 3 - protein, 143 aa, synthetic construct
  REGION 1..143: note = Recombinant MYDGF variant +G
GVSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKASRTEL

SEQ ID NO: 4 - protein, 141 aa, synthetic construct
  REGION 1..141: note = Recombinant MYDGF variant -V
SEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKASRTEL

SEQ ID NO: 5 - protein, 173 aa, Homo sapiens
  REGION 1..173: note = MYDGF with signal peptide
MAAPSGGWNGVGASLWAALLLGAVALRPAEAVSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKASRTEL

SEQ ID NO: 6 - protein, 142 aa, Homo sapiens
  REGION 1..142: note = Mature MYDGF after processing
VSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKASRTEL

SEQ ID NO: 7 - DNA, 4975 bp, synthetic construct
  misc_feature 1..4975: note = Expression vector encoding recombinant MYDGF variant +A
  source 1..4975: mol_type = other DNA
agatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagcggtacccgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctctgagcccgaagtggcgagcccgatcttccccatcggtgatgtcggcgatataggcgccagcaaccgcacctgtggcgccggtgatgccggccacgatgcgtccggcgtagaggatcgagatcgatctcatgatcccgtcgacgaaattaatacgactcactataggggaattgtgagcggataacaattcccctctacaaataattttgtttaactttaagaaggagatatacatatggcagtttctgaacctacgacggttgcttttgatgttcgcccgggtggtgttgtacacagctttagccacaacgtcggtccgggcgacaagtatacgtgcatgttcacgtacgcaagccagggcggcaccaatgaacaatggcagatgagcctgggcacttccgaggatcatcaacactttacctgtaccatctggcgtccgcaaggtaagtcgtacctgtacttcacccagttcaaagccgaagtgcgcggtgcggaaatcgagtatgccatggcgtatagcaaagccgcgttcgagcgtgagagcgacgtgccattgaaaaccgaagaatttgaagtgaccaagaccgcagtggcgcatcgtccgggtgcgttcaaagctgagctgagcaaactggtcattgtcgcgaaggcaagccgtaccgagctgtaataggaattcgctagtctagactcgagatcactagtaccggtccatggcagcccgggtaaagcaccgcaatgatggagaattagatccggctgctaacaaagcccgaaaggaagctgagttggctgctgccaccgctgagcaataactagcataaccccgcggggcctcttcgggggtctcgcggggttttttgctgaaagagcttcaaataaaacgaaaggctcagtcgaaagactgggcctttcgttttatctgttgtttgtcgctgcgccgcactcgcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggcaggtgggatccattgaaatacaggaacgcacgctggatggcccttcgctgggatggtgaaaccatgaaaaatggcagcttcagtggattaagtgggggtaatgtggcctgtaccctctggttgcataggtattcatacggttaaaatttatcaggcgcgatcgcggcagtttttcgggtggtttgttgccatttttacctgtctgctgccgtgatcgcgctgaacgcgttttagcggtgcgtacaattaagggattatggtaaatccacttactgtctgccctcgtagccataagcttcaggtggcacttttcggggaaatgtgcgcggaacccctatttgtttatttttctaaatacattcaaatatgtatccgctcgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttaccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgtagatctacatgaccaaaatcccttaacgtgagttttcgttccactgagcgtcagaccccgtagaaa

SEQ ID NO: 8 - DNA, 4975 bp, synthetic construct
  misc_feature 1..4975: note = Expression vector encoding recombinant MYDGF variant +S
  source 1..4975: mol_type = other DNA
agatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagcggtacccgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctctgagcccgaagtggcgagcccgatcttccccatcggtgatgtcggcgatataggcgccagcaaccgcacctgtggcgccggtgatgccggccacgatgcgtccggcgtagaggatcgagatcgatctcatgatcccgtcgacgaaattaatacgactcactataggggaattgtgagcggataacaattcccctctacaaataattttgtttaactttaagaaggagatatacatatgagcgtttctgaacctacgacggttgcttttgatgttcgcccgggtggtgttgtacacagctttagccacaacgtcggtccgggcgacaagtatacgtgcatgttcacgtacgcaagccagggcggcaccaatgaacaatggcagatgagcctgggcacttccgaggatcatcaacactttacctgtaccatctggcgtccgcaaggtaagtcgtacctgtacttcacccagttcaaagccgaagtgcgcggtgcggaaatcgagtatgccatggcgtatagcaaagccgcgttcgagcgtgagagcgacgtgccattgaaaaccgaagaatttgaagtgaccaagaccgcagtggcgcatcgtccgggtgcgttcaaagctgagctgagcaaactggtcattgtcgcgaaggcaagccgtaccgagctgtaataggaattcgctagtctagactcgagatcactagtaccggtccatggcagcccgggtaaagcaccgcaatgatggagaattagatccggctgctaacaaagcccgaaaggaagctgagttggctgctgccaccgctgagcaataactagcataaccccgcggggcctcttcgggggtctcgcggggttttttgctgaaagagcttcaaataaaacgaaaggctcagtcgaaagactgggcctttcgttttatctgttgtttgtcgctgcgccgcactcgcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggcaggtgggatccattgaaatacaggaacgcacgctggatggcccttcgctgggatggtgaaaccatgaaaaatggcagcttcagtggattaagtgggggtaatgtggcctgtaccctctggttgcataggtattcatacggttaaaatttatcaggcgcgatcgcggcagtttttcgggtggtttgttgccatttttacctgtctgctgccgtgatcgcgctgaacgcgttttagcggtgcgtacaattaagggattatggtaaatccacttactgtctgccctcgtagccataagcttcaggtggcacttttcggggaaatgtgcgcggaacccctatttgtttatttttctaaatacattcaaatatgtatccgctcgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttaccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgtagatctacatgaccaaaatcccttaacgtgagttttcgttccactgagcgtcagaccccgtagaaa

SEQ ID NO: 9 - DNA, 4975 bp, synthetic construct
  misc_feature 1..4975: note = Expression vector encoding recombinant MYDGF variant +G
  source 1..4975: mol_type = other DNA
agatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagcggtacccgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctctgagcccgaagtggcgagcccgatcttccccatcggtgatgtcggcgatataggcgccagcaaccgcacctgtggcgccggtgatgccggccacgatgcgtccggcgtagaggatcgagatcgatctcatgatcccgtcgacgaaattaatacgactcactataggggaattgtgagcggataacaattcccctctacaaataattttgtttaactttaagaaggagatatacatatgggtgtttctgaacctacgacggttgcttttgatgttcgcccgggtggtgttgtacacagctttagccacaacgtcggtccgggcgacaagtatacgtgcatgttcacgtacgcaagccagggcggcaccaatgaacaatggcagatgagcctgggcacttccgaggatcatcaacactttacctgtaccatctggcgtccgcaaggtaagtcgtacctgtacttcacccagttcaaagccgaagtgcgcggtgcggaaatcgagtatgccatggcgtatagcaaagccgcgttcgagcgtgagagcgacgtgccattgaaaaccgaagaatttgaagtgaccaagaccgcagtggcgcatcgtccgggtgcgttcaaagctgagctgagcaaactggtcattgtcgcgaaggcaagccgtaccgagctgtaataggaattcgctagtctagactcgagatcactagtaccggtccatggcagcccgggtaaagcaccgcaatgatggagaattagatccggctgctaacaaagcccgaaaggaagctgagttggctgctgccaccgctgagcaataactagcataaccccgcggggcctcttcgggggtctcgcggggttttttgctgaaagagcttcaaataaaacgaaaggctcagtcgaaagactgggcctttcgttttatctgttgtttgtcgctgcgccgcactcgcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggcaggtgggatccattgaaatacaggaacgcacgctggatggcccttcgctgggatggtgaaaccatgaaaaatggcagcttcagtggattaagtgggggtaatgtggcctgtaccctctggttgcataggtattcatacggttaaaatttatcaggcgcgatcgcggcagtttttcgggtggtttgttgccatttttacctgtctgctgccgtgatcgcgctgaacgcgttttagcggtgcgtacaattaagggattatggtaaatccacttactgtctgccctcgtagccataagcttcaggtggcacttttcggggaaatgtgcgcggaacccctatttgtttatttttctaaatacattcaaatatgtatccgctcgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttaccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgtagatctacatgaccaaaatcccttaacgtgagttttcgttccactgagcgtcagaccccgtagaaa

SEQ ID NO: 10 - DNA, 4969 bp, synthetic construct
  misc_feature 1..4969: note = Expression vector encoding recombinant MYDGF variant -V
  source 1..4969: mol_type = other DNA
agatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagcggtacccgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctctgagcccgaagtggcgagcccgatcttccccatcggtgatgtcggcgatataggcgccagcaaccgcacctgtggcgccggtgatgccggccacgatgcgtccggcgtagaggatcgagatcgatctcatgatcccgtcgacgaaattaatacgactcactataggggaattgtgagcggataacaattcccctctacaaataattttgtttaactttaagaaggagatatacatatgtctgaacctacgacggttgcttttgatgttcgcccgggtggtgttgtacacagctttagccacaacgtcggtccgggcgacaagtatacgtgcatgttcacgtacgcaagccagggcggcaccaatgaacaatggcagatgagcctgggcacttccgaggatcatcaacactttacctgtaccatctggcgtccgcaaggtaagtcgtacctgtacttcacccagttcaaagccgaagtgcgcggtgcggaaatcgagtatgccatggcgtatagcaaagccgcgttcgagcgtgagagcgacgtgccattgaaaaccgaagaatttgaagtgaccaagaccgcagtggcgcatcgtccgggtgcgttcaaagctgagctgagcaaactggtcattgtcgcgaaggcaagccgtaccgagctgtaataggaattcgctagtctagactcgagatcactagtaccggtccatggcagcccgggtaaagcaccgcaatgatggagaattagatccggctgctaacaaagcccgaaaggaagctgagttggctgctgccaccgctgagcaataactagcataaccccgcggggcctcttcgggggtctcgcggggttttttgctgaaagagcttcaaataaaacgaaaggctcagtcgaaagactgggcctttcgttttatctgttgtttgtcgctgcgccgcactcgcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggcaggtgggatccattgaaatacaggaacgcacgctggatggcccttcgctgggatggtgaaaccatgaaaaatggcagcttcagtggattaagtgggggtaatgtggcctgtaccctctggttgcataggtattcatacggttaaaatttatcaggcgcgatcgcggcagtttttcgggtggtttgttgccatttttacctgtctgctgccgtgatcgcgctgaacgcgttttagcggtgcgtacaattaagggattatggtaaatccacttactgtctgccctcgtagccataagcttcaggtggcacttttcggggaaatgtgcgcggaacccctatttgtttatttttctaaatacattcaaatatgtatccgctcgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttaccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgtagatctacatgaccaaaatcccttaacgtgagttttcgttccactgagcgtcagaccccgtagaaa

SEQ ID NO: 11 - DNA, 438 bp, synthetic construct
  misc_feature 1..438: note = DNA insert encoding recombinant modified MYDGF variant +A
  source 1..438: mol_type = other DNA
atggcagtttctgaacctacgacggttgcttttgatgttcgcccgggtggtgttgtacacagctttagccacaacgtcggtccgggcgacaagtatacgtgcatgttcacgtacgcaagccagggcggcaccaatgaacaatggcagatgagcctgggcacttccgaggatcatcaacactttacctgtaccatctggcgtccgcaaggtaagtcgtacctgtacttcacccagttcaaagccgaagtgcgcggtgcggaaatcgagtatgccatggcgtatagcaaagccgcgttcgagcgtgagagcgacgtgccattgaaaaccgaagaatttgaagtgaccaagaccgcagtggcgcatcgtccgggtgcgttcaaagctgagctgagcaaactggtcattgtcgcgaaggcaagccgtaccgagctgtaatag

SEQ ID NO: 12 - DNA, 438 bp, synthetic construct
  misc_feature 1..438: note = DNA insert encoding recombinant modified MYDGF variant +S
  source 1..438: mol_type = other DNA
atgagcgtttctgaacctacgacggttgcttttgatgttcgcccgggtggtgttgtacacagctttagccacaacgtcggtccgggcgacaagtatacgtgcatgttcacgtacgcaagccagggcggcaccaatgaacaatggcagatgagcctgggcacttccgaggatcatcaacactttacctgtaccatctggcgtccgcaaggtaagtcgtacctgtacttcacccagttcaaagccgaagtgcgcggtgcggaaatcgagtatgccatggcgtatagcaaagccgcgttcgagcgtgagagcgacgtgccattgaaaaccgaagaatttgaagtgaccaagaccgcagtggcgcatcgtccgggtgcgttcaaagctgagctgagcaaactggtcattgtcgcgaaggcaagccgtaccgagctgtaatag

SEQ ID NO: 13 - DNA, 438 bp, synthetic construct
  misc_feature 1..438: note = DNA insert encoding recombinant modified MYDGF variant +G
  source 1..438: mol_type = other DNA
atgggtgtttctgaacctacgacggttgcttttgatgttcgcccgggtggtgttgtacacagctttagccacaacgtcggtccgggcgacaagtatacgtgcatgttcacgtacgcaagccagggcggcaccaatgaacaatggcagatgagcctgggcacttccgaggatcatcaacactttacctgtaccatctggcgtccgcaaggtaagtcgtacctgtacttcacccagttcaaagccgaagtgcgcggtgcggaaatcgagtatgccatggcgtatagcaaagccgcgttcgagcgtgagagcgacgtgccattgaaaaccgaagaatttgaagtgaccaagaccgcagtggcgcatcgtccgggtgcgttcaaagctgagctgagcaaactggtcattgtcgcgaaggcaagccgtaccgagctgtaatag

SEQ ID NO: 14 - DNA, 432 bp, synthetic construct
  misc_feature 1..432: note = DNA insert encoding recombinant modified MYDGF variant -V
  source 1..432: mol_type = other DNA
atgtctgaacctacgacggttgcttttgatgttcgcccgggtggtgttgtacacagctttagccacaacgtcggtccgggcgacaagtatacgtgcatgttcacgtacgcaagccagggcggcaccaatgaacaatggcagatgagcctgggcacttccgaggatcatcaacactttacctgtaccatctggcgtccgcaaggtaagtcgtacctgtacttcacccagttcaaagccgaagtgcgcggtgcggaaatcgagtatgccatggcgtatagcaaagccgcgttcgagcgtgagagcgacgtgccattgaaaaccgaagaatttgaagtgaccaagaccgcagtggcgcatcgtccgggtgcgttcaaagctgagctgagcaaactggtcattgtcgcgaaggcaagccgtaccgagctgtaatag

SEQ ID NO: 15 - protein, 144 aa, synthetic construct
  REGION 1..144: note = Recombinant MYDGF variant +A with Met
MAVSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKASRTEL

SEQ ID NO: 16 - protein, 144 aa, synthetic construct
  REGION 1..144: note = Recombinant MYDGF variant +S with Met
MSVSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKASRTEL

SEQ ID NO: 17 - protein, 144 aa, synthetic construct
  REGION 1..144: note = Recombinant MYDGF variant +G with Met
MGVSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKASRTEL

SEQ ID NO: 18 - protein, 142 aa, synthetic construct [sequence omitted]

(19) United States
(12) Patent Application Publication
(10) Pub. No.: US 2025/0333459 A1
(43) Pub. Date: Oct. 30, 2025

(54) RECOMBINANT EXPRESSION OF MYELOID-DERIVED GROWTH FACTOR

(71) Applicant: BOEHRINGER INGELHEIM INTERNATIONAL GMBH, Ingelheim Am Rhein (DE)

(72) Inventors: Matthias BERKEMEYER, Ingelheim Am Rhein (DE); Anton PEKCEC, Ingelheim Am Rhein (DE); Priyanka GUPTA, Ridgefield, CT (US); Jon Reed, Ridgefield, CT (US); Mortimer KORF-KLINGEBIEL, Duderstadt (DE); Cornelia WALTHER, Ingelheim Am Rhein (DE); Kai Christoph WOLLERT, Hannover (DE)

(21) Appl. No.: 18/871,433

(22) PCT Filed: Jun. 3, 2023
(86) PCT No.: PCT/EP2023/064899

§ 371 Date: Dec. 3, 2024

Related U.S. Application Data
(60) Provisional application No. 63/348,872, filed on Jun. 3, 2022.
(60) Provisional application No. 63/350,317, filed on Jun. 8, 2022.

(51) Int. Cl. C07K 14/475; A61K 38/18; A61P 9/10; C12N 15/70

(52) U.S. Cl. CPC C07K 14/475; A61K 38/18; A61P 9/10; C12N 15/70

(57) ABSTRACT

The present invention generally relates to the field of recombinant gene expression in host cells. In particular, the invention relates to a recombinant human myeloid-derived growth factor (MYDGF) protein that exhibits a minimal degree of degradation upon expression in a host cell. The recombinant protein is therefore highly suitable for medical use, in particular for treating heart tissue damage and preventing cell death in myocardial tissue. The invention also provides a nucleic acid which encodes the recombinant protein and a host cell that expresses the recombinant protein. The invention also provides a method for producing the recombinant protein in a host cell.

The present invention generally relates to the field of recombinant gene expression in host cells. In particular, the invention relates to a recombinant human myeloid-derived growth factor (MYDGF) protein that exhibits a minimal degree of degradation upon expression in a host cell. The recombinant protein is therefore highly suitable for medical use, in particular for treating heart tissue damage and preventing cell death in myocardial tissue. The invention also provides a nucleic acid which encodes the recombinant protein and a host cell that expresses the recombinant protein. The invention also provides a method for producing the recombinant protein in a host cell.

### BACKGROUND OF THE INVENTION

Acute myocardial infarction (MI) still is one of the major causes for morbidity and mortality worldwide. Acute MI is mediated by a thrombotic occlusion of a coronary artery, which leads to progressive cell death in the non-perfused tissue. This triggers an inflammatory response, which leads to scar formation and loss of viable tissue. Severe alteration of tissue architecture in the left ventricle can cause chamber dilatation, contractile dysfunction and heart failure. A protein named myeloid-derived growth factor (MYDGF) has shown to improve tissue repair and heart function in rodent models of MI. In comparison to wild-type mice, MYDGF-deficient mice develop larger infarct scars and more severe contractile dysfunctions. It was found that treatment with recombinant MYDGF is able to protect cardiomyocytes from cell deaths and repair the heart after acute MI. The development of a protein-based therapy would be a promising approach for cardiac repair and potentially also for ischemic repair in other tissues (Ebenhoch et al., 2019; Polten et al., 2019; Botnov et al., 2018, Korf-Klingebiel et al., 2015, WO 2014/111458).

At present, small amounts of recombinant human MYDGF are produced by expression in human or mammalian expression systems, such as HEK-293T or CHO cells. However, the larger-scale production of recombinant human MYDGF (rhMYDGF) by mammalian cell expression systems is associated with high costs which render the production unattractive from the economical perspective. Attempts have been made to produce rhMYDGF in bacterial expression systems. Zhao et al., 2020 describe the expression of soluble rhMYDGF with a C-terminal Histag in E. coli. The tagged protein differs from mature human wild-type protein in 9 additional amino acids, thereby having a significantly higher molecular weight. Although the authors speculate in that publication that the expression system might be used for producing rhMYDGF for clinical use, the His-Tag would pose a significant antigenicity risk when administered to a human patient.

It is also known that a heterologous expression of rhMYDGF is associated with considerable degradation problems. After recombinant expression of the protein, one or more amino acids located at the N-terminus are degraded which gives rise to protein fragments having a smaller molecular weight (MW) compared to the full-length protein. For example, Zhao et al., 2020 describe that the final expression product comprises not only the full-length rhMYDGF protein having a MW of 17032 Da, but also a degradation product with a MW of about 16900 Da. The ratio of target protein to degraded protein in Zhao et al., 2020 is approximately 10:1, as can be taken from the high performance liquid chromatography-mass spectrometry (HPLC-MS) data shown in Fig. 2 of the Zhao publication. Thus, the impurity by the degradation product is quite significant and not acceptable for a protein that is intended for systemic medical use.

In view of the prior art set out above, it is an object of the present invention to provide a recombinant protein with MYDGF activity that
(i) exhibits no degradation or only minimal degradation upon expression in a heterologous expression system;
(ii) can be produced with a minimal degree of potentially adverse process-derived post translational modifications, such as carbamoylation and gluconoylation which may be detrimental for the intended clinical use of the protein;
(iii) has the secondary and tertiary structure of the native human MYDGF protein;
(iv) has a high biologically activity;
(v) is associated with a low risk for primary sequence derived antigenic epitopes other than human MYDGF thereby creating a minimal risk for anti-drug antibodies;
(vi) can be produced in a prokaryotic expression system to provide a non-glycosylated product;
(vii) can be produced in an amount of more than 0.5 g protein per 100 g cells and is scalable to more than 100 g, preferably more than 200 g, more preferably more than 300 g protein per batch.

Not all of the objectives will be realized by all embodiments of the invention. The scope of the invention is defined by the claims. It is however preferred to meet 2, 3, 4, 5, or 6 of the aforementioned objectives of the invention.

### SUMMARY OF THE INVENTION

In a first aspect, the invention relates to a method for the recombinant expression of a MYDGF protein in a host cell.

In a second aspect, the invention relates to a composition which is obtainable from the method of the first aspect of the invention.

In a third aspect, the invention relates to the use of a composition of the second aspect of the invention for the preparation of a pharmaceutical composition.

In a fourth aspect, the invention relates to a pharmaceutical composition comprising a composition of the second aspect of the invention.

In a fifth aspect, the invention relates to a pharmaceutical composition of the fourth aspect of the invention for use as a medicament.

In a sixth aspect, the invention relates to a composition of the second aspect of the invention or a pharmaceutical composition of the fourth aspect of the invention for use in a method of (i) treating or preventing a disease or condition selected form the group consisting of injury, wounding, ischemia, reperfusion injury, trauma, mechanical overload, intoxication, surgery, primary or acquired cardiomyopathy, postischemic contractile dysfunction, myocardial infarction, preferably acute myocardial infarction, angina pectoris, heart failure, inflammation of the heart, heart insufficiency, hypertrophy, and fibrosis; (ii) promoting or improving heart tissue regeneration, cardiomyocyte proliferation, neovascularisation, heart function or left ventricular systolic function after myocardial infarction; (iii) protecting cardiomyocyte from death, e.g. through apoptosis or necrosis; or (iv) decreasing infarct size after myocardial infarction, preferably acute myocardial infarction.

In a seventh aspect, the invention relates to a protein having the amino acid sequence of SEQ ID NO:1 or SEQ ID NO:2, and preferably SEQ ID NO:1.

In an eighth aspect, the invention relates to a nucleic acid encoding a protein of the seventh aspect of the invention.

In a ninth aspect, the invention relates to a vector comprising the nucleic acid of the eighth aspect of the invention.

In a tenth aspect, the invention relates to a host cell comprising a protein of the seventh aspect of the invention, a nucleic acid of the eighth aspect of the invention, or a vector of the ninth aspect of the invention.

In an eleventh aspect, the invention relates to a pharmaceutical composition comprising the protein of the seventh aspect of the invention.

In a twelfth aspect, the invention relates to a protein of the seventh aspect of the invention or a pharmaceutical composition of the eleventh aspect of the invention for use as a medicament.

In a thirteenth aspect, the invention relates to a protein of the seventh aspect of the invention or a pharmaceutical composition of the eleventh aspect of the invention for use in a method of (i) treating or preventing a disease or condition selected from the group consisting of injury, wounding, ischemia, reperfusion injury, trauma, mechanical overload, intoxication, surgery, primary or acquired cardiomyopathy, postischemic contractile dysfunction, myocardial infarction, preferably acute myocardial infarction, angina pectoris, heart failure, inflammation of the heart, heart insufficiency, hypertrophy, and fibrosis; (ii) promoting or improving heart tissue regeneration, cardiomyocyte proliferation, neovascularisation, heart function or left ventricular systolic function after myocardial infarction; (iii) protecting cardiomyocyte from death, e.g. through apoptosis or necrosis; or (iv) decreasing infarct size after myocardial infarction, preferably acute myocardial infarction.

In a fourteenth aspect, the invention relates to a method for the recombinant expression of a protein of the seventh aspect of the invention in a host cell.

In a fifteenth aspect, the invention relates to a composition which is obtainable from the method of the fourteenth aspect of the invention.

Finally, in a sixteenth aspect, the invention relates to the use of a host cell of the tenth aspect of the invention for the recombinant expression of a MYDGF protein.

### DETAILED DESCRIPTION OF THE INVENTION

The present invention provides proteins having MYDGF activity and exerting a minimal degree of degradation and process-derived post translational modifications upon recombinant expression in a host cell. The invention also provides a method for producing these proteins in large amounts in a cell-based expression system. The method requires significantly reduced purification efforts for providing a homogeneous protein composition that is suitable for being formulated into a pharmaceutical product. If reference is made to SEQ ID NO:1 or SEQ ID NO:2 hereinafter, it must be understood that SEQ ID NO:1 is the preferred alternative.

The proteins disclosed herein in the context with the present invention are depicted in SEQ ID NO:1 and SEQ ID NO:2. Both proteins consist of 143 amino acid building blocks and comprise the complete amino acid sequence of the mature human MYDGF protein. The native human MYDGF protein is expressed as a precursor protein with an N-terminal signal peptide of 31 amino acids and a C-terminal KDEL-like endoplasmic reticulum (ER) retention sequence. The sequence of the MYDGF precursor protein of 173 amino acids is set forth herein as SEQ ID NO:5. Upon cleavage of the N-terminal signal peptide, the mature MYDGF is released. The sequence of the mature human MYDGF protein consists of 142 amino acids and is set forth herein as SEQ ID NO:6. The proteins of the invention differ from mature MYDGF only in a single amino acid that has been added to their N-terminus. Hence, these proteins can be regarded as recombinant variants of the native human MYDGF protein. The protein of SEQ ID NO:1 comprises an additional alanine residue at its N-terminus, which is not present in the mature human MYDGF protein. This variant is referred to as “[+A]” or “the [+A] variant” herein below. The protein of SEQ ID NO:2 differs from the mature human MYDGF protein by an additional serine residue at its N-terminus. This variant is referred to herein as “[+S]” or “the [+S] variant”.

The proteins are associated with a particularly low risk of comprising antigenic epitopes that are not derived from human MYDGF. Accordingly, the proteins of the invention exert a minimal risk for generating anti-drug antibodies. The differences between the proteins of the invention and native human MYDGF protein reside in a single amino acid which means that the region added to the native protein is too small to give rise to new epitopes. The absence of anti-drug antibodies renders the proteins of the invention highly suitable for being used for therapeutic purposes. Preferably, the administration of the proteins to humans will generate only a minimal level of anti-drug antibodies or antibodies directed to endogenous MYDGF, and more preferably not at all.

As can be seen from the below Examples, the proteins of the invention exhibit a low degree of chemical and post-translational modifications upon expression in a cell-based expression system, i.e. in an expression system that uses eukaryotic or prokaryotic cells for the recombinant production of the protein. The effective reduction of chemical and post-translational modifications during the production of proteins for pharmaceutical applications is of fundamental importance. In particular, the proteins of the invention are characterized by a low degree of carbamoylation and gluconoylation. As used herein, carbamoylation is a non-enzymatic reaction in which a carbamoyl moiety is added to a protein, peptide or amino acid. After expression in a cell-based expression system, isolation of the proteins from inclusion bodies and protein refolding, carbamoylation preferably occurs in less than 6.0% (w/w) of the total protein of SEQ ID NO:1, more preferably in less than 5.5% (w/w), less than 5.0% (w/w), less than 4.5% (w/w), or less than 4.0% (w/w), of the total protein having the amino acid sequence of SEQ ID NO:1. Similarly, carbamoylation preferably occurs in less than 6.0% (w/w) of the total protein of SEQ ID NO:2, more preferably in less than 5.5% (w/w), less than 5.0% (w/w), less than 4.5% (w/w), or less than 4.0% (w/w), of the total protein having the amino acid sequence of SEQ ID NO:2 after expression of the protein in a cell-based expression system, isolation of the proteins from inclusion bodies and protein refolding. In general, a level of carbamoylation of less than 6.0% (w/w) is acceptable and does not pose a risk for pharmaceutical applications.

In addition, the proteins disclosed herein are characterized by a low degree of gluconoylation. The gluconoylation of recombinantly expressed protein is regularly observed in bacterial host cells, such as in cells of E. coli BL21(DE3). This modification results from the formation of 6-phosphogluconolactone (6-PGLac), a compound that is produced by the enzyme glucose-6-phosphate dehydrogenase. With the proteins of the invention, gluconoylation preferably occurs after expression in a cell-based expression system, isolation of the protein from inclusion bodies and protein refolding only in less than 4.0% (w/w) of the total protein of SEQ ID NO:1, more preferably in less than 3.5% (w/w), less than 3.0% (w/w), less than 2.5% (w/w), or less than 2.0% (w/w), of the total protein of SEQ ID NO:1. Similarly, carbamoylation preferably occurs in less than 4.0% (w/w) of the total protein of SEQ ID NO:2, more preferably in less than 3.5% (w/w), less than 3.0% (w/w), less than 2.5% (w/w), or less than 2.0% (w/w), of the total protein of SEQ ID NO:2. In general, a level of gluconoylation of less than 4% is acceptable and does not pose a risk for pharmaceutical applications.

A further embodiment of the invention relates to a protein having the amino acid sequence of SEQ ID NO:1 and moreover having a spectrum in the two-dimensional nuclear magnetic resonance spectroscopy (2D-NMR) which is essentially identical to the one shown in the below Table 1. In particular, the protein has an NMR spectrum that comprises at least 2 of the ¹H and/or ¹⁵N peaks, and preferably at least 4, at least 6, at least 8, at least 10, at least 12, at least 14, at least 16, at least 18, at least 20, at least 22, at least 24, at least 26, at least 28, at least 30, at least 32, at least 34, at least 36, at least 38, at least 40, at least 42, at least 44, at least 46, at least 48, at least 50, at least 52, at least 54, at least 56, at least 58, at least 60, at least 62, at least 64, at least 66, at least 68, at least 70, at least 72, at least 74, at least 76, at least 78, at least 80, at least 82, at least 84, at least 86, at least 88, at least 90, at least 92, at least 94, at least 96, at least 98, at least 100, at least 102, at least 104, at least 106, at least 108, at least 110, at least 112, at least 114, at least 116, at least 118, at least 120, at least 122, at least 124, at least 126, at least 128, at least 130, at least 132, or at least 134 peaks of the ¹H and/or ¹⁵N peaks in Table 1 when analysing a sample of 8.5 mg/ml of the respective MYDGF protein in 50 mM sodium phosphate buffer at pH 7.4 containing 50 mM sodium chloride and 9% (v/v) D₂O.

Most preferably, the protein has an NMR spectrum comprising or consisting of all 136 of the ¹H and/or ¹⁵N peaks set forth in Table 1 when analysing a sample of 8.5 mg/ml of the respective MYDGF protein in 50 mM sodium phosphate buffer at pH 7.4 containing 50 mM sodium chloride and 9% (v/v) D₂O. Thus, the protein has the secondary and tertiary structure of the native human MYDGF protein.

In yet another embodiment, the present invention relates to a protein which is folded such that more than 70%, and preferably more than 80%, more than 90%, or more than 95%, of the ¹H and/or ¹⁵N peaks in the 2D-NMR map result in combined chemical shift deviation (CCSD) values below 0.01 ppm when compared to the corresponding peaks in Table 1. The CCSD is calculated according to the following formula (Brinson et al. 2019):

$$CCSD = \sqrt{\frac{{( {δ}_{H} - {δ}_{Href} )}^{2} + {( \frac{{δ}_{N} - {δ}_{Nref}}{10} )}^{2}}{2}}$$

in which δ_{H} and δ_{N} represent the ¹H and ¹⁵N chemical shifts of a given cross peak, respectively, and δ_{Href} and δ_{Nref} represent the ¹H and ¹⁵N reference chemical shifts for the same cross peak. If more than 70%, and preferably more than 80%, more than 90%, or more than 95%, of the ¹H and/or ¹⁵N peaks in the 2D-NMR map lead to CCSD values below 0.01 ppm, the protein folding is very similar to the one observed for the [+A] variant in Table 1. Specifically, if more than 90%, or more than 95% of the ¹H and/or ¹⁵N peaks in the 2D-NMR map exert CCSD values below 0.01 ppm, the protein folding is almost identical to the one observed for the [+A] variant in Table 1.

TABLE 1
NMR spectrum [+A]

| | 1H | 15N |
|---|---|---|
| | chemical shift | chemical shift |
| Peak No. | (ppm) | (ppm) |
| 1 | 10.37 | 129.76 |
| 2 | 10.14 | 129.36 |
| 3 | 9.60 | 129.47 |
| 4 | 9.63 | 128.50 |
| 5 | 9.49 | 128.58 |
| 6 | 9.13 | 129.31 |
| 7 | 8.87 | 129.18 |
| 8 | 9.04 | 128.69 |
| 9 | 9.02 | 128.32 |
| 10 | 9.48 | 126.84 |
| 11 | 9.20 | 126.47 |
| 12 | 8.95 | 126.81 |
| 13 | 8.79 | 127.05 |
| 14 | 8.48 | 127.45 |
| 15 | 8.14 | 128.38 |
| 16 | 7.84 | 129.41 |
| 17 | 8.81 | 125.63 |
| 18 | 8.70 | 125.75 |
| 19 | 8.25 | 126.85 |
| 20 | 9.20 | 125.74 |
| 21 | 9.30 | 124.99 |
| 22 | 9.03 | 124.81 |
| 23 | 8.86 | 124.98 |
| 24 | 8.81 | 124.67 |
| 25 | 8.20 | 125.80 |
| 26 | 7.88 | 125.80 |
| 27 | 8.44 | 124.83 |
| 28 | 8.36 | 124.50 |
| 29 | 7.72 | 124.72 |
| 30 | 7.60 | 123.64 |
| 31 | 7.63 | 122.68 |
| 32 | 7.42 | 122.77 |
| 33 | 7.91 | 122.40 |
| 34 | 8.38 | 122.53 |
| 35 | 8.37 | 123.27 |
| 36 | 8.50 | 123.58 |
| 37 | 8.60 | 123.33 |
| 38 | 8.65 | 123.98 |
| 39 | 8.77 | 123.34 |
| 40 | 8.72 | 122.90 |
| 41 | 8.89 | 122.88 |
| 42 | 9.00 | 121.91 |
| 43 | 8.27 | 121.54 |
| 44 | 9.07 | 122.08 |
| 45 | 9.12 | 121.13 |
| 46 | 9.39 | 121.60 |
| 47 | 9.33 | 120.96 |
| 48 | 9.41 | 120.69 |
| 49 | 9.03 | 120.73 |
| 50 | 8.37 | 120.75 |
| 51 | 8.49 | 120.29 |
| 55 | 9.08 | 119.50 |
| 56 | 8.82 | 119.49 |
| 57 | 8.77 | 119.60 |
| 58 | 8.45 | 119.33 |
| 59 | 7.99 | 119.50 |
| 60 | 8.06 | 120.13 |
| 61 | 8.16 | 120.20 |
| 62 | 8.30 | 119.98 |
| 63 | 8.30 | 119.68 |
| 64 | 8.30 | 118.69 |
| 65 | 7.66 | 121.40 |
| 66 | 7.57 | 121.52 |
| 67 | 7.22 | 121.78 |
| 68 | 7.17 | 121.00 |
| 69 | 7.47 | 119.60 |
| 70 | 7.34 | 119.67 |
| 71 | 7.26 | 119.24 |
| 72 | 7.17 | 119.38 |
| 73 | 7.33 | 118.42 |
| 74 | 6.68 | 120.50 |
| 75 | 6.15 | 117.12 |
| 76 | 7.71 | 117.75 |
| 77 | 8.02 | 117.15 |
| 78 | 8.09 | 117.30 |
| 79 | 8.18 | 117.90 |
| 80 | 8.19 | 117.22 |
| 81 | 8.42 | 118.42 |
| 82 | 8.52 | 117.35 |
| 83 | 8.56 | 116.70 |
| 84 | 8.26 | 116.36 |
| 85 | 8.67 | 117.13 |
| 86 | 8.70 | 116.53 |
| 87 | 8.77 | 117.66 |
| 88 | 8.77 | 116.10 |
| 89 | 8.28 | 115.50 |
| 90 | 8.22 | 115.01 |
| 91 | 7.91 | 115.17 |
| 92 | 7.88 | 115.81 |
| 93 | 7.66 | 115.39 |
| 94 | 7.65 | 113.72 |
| 95 | 8.62 | 114.63 |
| 96 | 8.59 | 113.55 |
| 97 | 9.94 | 115.99 |
| 98 | 9.26 | 118.39 |
| 99 | 9.08 | 118.84 |
| 100 | 8.73 | 116.99 |
| 101 | 7.23 | 115.12 |
| 102 | 6.64 | 115.15 |
| 103 | 6.67 | 113.72 |
| 104 | 7.53 | 112.93 |
| 105 | 6.82 | 112.88 |
| 106 | 6.68 | 112.93 |
| 107 | 7.38 | 112.88 |
| 108 | 7.36 | 112.79 |
| 109 | 7.77 | 111.93 |
| 110 | 7.97 | 112.54 |
| 111 | 8.40 | 112.70 |
| 112 | 8.33 | 111.68 |
| 113 | 8.48 | 111.24 |
| 114 | 7.36 | 111.36 |
| 115 | 6.72 | 111.34 |
| 116 | 7.29 | 110.79 |
| 117 | 6.58 | 110.76 |
| 118 | 6.77 | 109.87 |
| 119 | 7.99 | 108.82 |
| 120 | 8.84 | 110.91 |
| 121 | 8.65 | 109.37 |
| 122 | 8.58 | 109.47 |
| 123 | 8.02 | 107.16 |
| 124 | 7.70 | 106.72 |
| 125 | 7.73 | 102.74 |
| 126 | 10.67 | 128.23 |
| 127 | 9.77 | 127.55 |
| 128 | 9.22 | 130.73 |
| 129 | 6.25 | 123.98 |
| 130 | 10.09 | 132.99 |
| 131 | 9.45 | 131.92 |
| 132 | 9.02 | 132.15 |
| 133 | 9.11 | 123.54 |
| 134 | 9.96 | 122.02 |
| 135 | 6.21 | 109.88 |
| 136 | 6.58 | 111.93 |

A further embodiment of the invention is a composition comprising a protein as described above, preferably a composition obtainable from recombinant expression in a bacterial expression system, such as the methods described in more detail below.

The composition of the invention may comprise a protein having the amino acid sequence of SEQ ID NO:1 along with variants thereof which are shorter in length and exert 100% sequence identity over their entire length with the amino acid sequence of SEQ ID NO:1, wherein the length is at least 100 amino acids with no gaps being allowed in the alignment. Within said composition, the ratio of the signal obtained from the protein according to SEQ ID NO:1 and the sum of signals obtained from said shorter variants, as determined by liquid chromatography mass spectrometry (LCMS) according to Tolonen et al (2011), is higher than 20, and preferably higher than 50, 75, 100, 125, 150, 175, 200, 225, 250, 275, 300, 325, 350, 375, 400, 425, and more preferably higher than 450. As can be seen from Table 15 below, the ratio of the signal obtained from the protein according to SEQ ID NO:1 and the signals obtained from shorter variants was found to be 466, as determined by LCMS according to Tolonen et al (2011). When calculating the ratio of the signal obtained from the protein according to SEQ ID NO:1 and the sum of signals obtained from said shorter variants, any carbamoylated or gluconoylated proteins are excluded. The percentage for the calculation of the ratio is based on the sum of the peak intensities of unmodified MYDGF protein as well as annotated post-translational modification (PTM) species of MYDGF in a deconvoluted intact mass spectrum of the MYDGF protein in the composition. The minimum requirements for mass spectrometry instrumentation and data processing are outlined in Example 4 below.

The composition of the invention may also comprise a protein having the amino acid sequence of SEQ ID NO:2 along with variants thereof which are shorter in length and exert 100% sequence identity over their entire length with the amino acid sequence of SEQ ID NO:2, wherein the length is at least 100 amino acids with no gaps being allowed in the alignment. Within said composition, the ratio of the signal obtained from the protein according to SEQ ID NO:2 and the sum of signals obtained from said shorter variants, as determined by LCMS according to Tolonen et al (2011), is higher than 20, and preferably higher than 50, 75, 100, 125, 150, and more preferably higher than 175 or 180. As can be seen from Table 15 below, the ratio of the signal obtained from the protein according to SEQ ID NO:2 and the signals obtained from shorter variants was found to be 186, as determined by LCMS according to Tolonen et al (2011). When calculating the ratio of the signal obtained from the protein according to SEQ ID NO:2 and the sum of signals obtained from said shorter variants, any carbamoylated or gluconoylated proteins are excluded. The percentage for the calculation of the ratio is based on the sum of the peak intensities of unmodified MYDGF protein as well as annotated post-translational modification (PTM) species of MYDGF in a deconvoluted intact mass spectrum of the MYDGF protein in the composition. The minimum requirements for mass spectrometry instrumentation and data processing are outlined in Example 4 below.

In one embodiment, less than 8%, and preferably less than 7%, less than 6% or less than 5% of the MYGDF proteins in the composition of the invention are carbamoylated, wherein the percentage is based on the sum of the peak intensities of unmodified MYDGF protein as well as annotated post-translational modification (PTM) species of MYDGF in a deconvoluted intact mass spectrum of the MYDGF protein in the composition. The minimum requirements for mass spectrometry instrumentation and data processing are outlined in Example 4 below.

In another embodiment, less than 6%, and preferably less than 5%, less than 4% or less than 3% of the MYDGF proteins in the composition of the invention are gluconylated, wherein the percentage is based on the sum of the peak intensities of unmodified MYDGF protein as well as annotated PTM species of MYDGF in a deconvoluted intact mass spectrum of the MYDGF protein in the composition. The minimum requirements for mass spectrometry instrumentation and data processing are outlined in Example 4 below.

According to another object of the invention, the composition of the invention preferably comprises a low amount of DNA that is derived from the host cell that was used for the production of the MYDGF protein. Preferably, the composition of the invention comprises less than 20 pg/mg, preferably less than 15 pg/mg, more preferably less than 10 pg/mg, and most preferably less than 5 pg host cell DNA per mg of the composition, such as less than 3 pg, less than 2 μg or less 1 pg host cell DNA per mg of the composition. Preferably, the presence of host cell DNA in the composition is determined by quantitative polymerase chain reaction (qPCR) such as real time qPCR.

According to another object of the invention, the composition of the invention preferably also comprises only a low amount of bacterial endotoxin that results from the production of the MYDGF protein in the bacterial host cells. Specifically, it is preferred that the composition comprises less than 0.2 EU per mg of the composition, and preferably less than 0.1 EU, less than 0.09 EU or 0.08 EU bacterial endotoxin per mg of the composition. Suitable methods for detecting the presence of bacterial endotoxin include the kinetic chromogenic method described in the current United States Pharmacopoeia (USP-NF 2021, issue 2, Chapter 85), the European Pharmacopoeia (10th edition 2021, 10.5, Chapter 2.6.14) and the Japanese Pharmacopoeia, Supplement II, JP 17th edition, 4.01).

According to another object of the invention, it is preferred that less than 8% (w/w), and preferably less than 7% (w/w), less than 6% (w/w) or less than 5% (w/w) of the proteins in the composition of the invention are carbamoylated. Similarly, it is preferred that less than 6% (w/w), and preferably less than 5% (w/w), less than 4% (w/w) or less than 3% (w/w), of the proteins in the composition of the invention are gluconoylated. It is particularly preferred that the composition of the invention comprises detectable amounts of carbamoylated proteins, wherein the amount of carbamoylated proteins in the composition is however less than 5% (w/w). Similarly, it is particularly preferred that the composition of the invention comprises detectable amounts of gluconoylated proteins, wherein the amount of gluconoylated proteins in the composition is however less than 5% (w/w).

The composition of the invention may comprise urea which results from the inclusion body solubilization and/or refolding step.

The composition of the invention preferably comprises no or only a low amount of protein aggregates that may result from the aggregation of protein molecules, such as di- tri- or oligomers of the MYDGF protein variant. Preferably, the composition comprises the MYDGF described above, i.e. the protein of SEQ ID NO:1 or SEQ ID NO:2, predominantly as a monomer. More preferably, the monomer content of the protein in the composition is 95% (w/w) or more, and even more preferably 96% (w/w) or more, 97% (w/w) or more, 98% (w/w) or more, or 99% (w/w) or more. Stated differently, the amount of aggregates of the protein in the composition is about 5% (w/w) or less, and even more preferably 4% (w/w) or less, 3% (w/w) or less, 2% (w/w) or less, or 1% (w/w) or less or is not detectable at all. The amount of protein monomers and aggregates is preferably measured by size exclusion chromatography (SEC), and more preferably by size exclusion high-performance liquid chromatography (SEC HPLC).

The composition of the invention preferably comprises a protein of 143 amino acids having the amino acid sequence of SEQ ID NO:1 or SEQ ID NO:2. The composition preferably comprises less than 5% (w/w), less than 4% (w/w), less than 3% (w/w), less than 2% (w/w) and preferably less than 1% (w/w) protein molecules that are shorter than 143 amino acids, as measured by LCMS.

The composition of the invention preferably comprises less than 5% (w/w), less than 4% (w/w), less than 3% (w/w), less than 2% (w/w) and more preferably less than 1% (w/w) protein molecules that differ from the amino acid sequence of SEQ ID NO:1 by either (i) the deletion of 1-4 amino acids at the N-terminus of SEQ ID NO:1, or (ii) the addition of a single amino acid at the N-terminus of SEQ ID NO:1, based on the overall weight of all ungluconoylated and uncarbamoylated proteins in said composition that comprise at least 100 contiguous amino acids of the sequence of SEQ ID NO:1, and determined by LCMS after reductive dimethylation (stable isotope dimethyl labelling, SIDL) according to Tolonen et al. (2011). The composition shown in Table 15 comprising the +A variant (SEQ ID NO:1) shows 0.2% of proteins according to (i) or (ii).

The composition of the invention preferably comprises less than 5% (w/w), less than 4% (w/w), less than 3% (w/w), less than 2% (w/w) and more preferably less than 1% (w/w) protein molecules that differ from the amino acid sequence of SEQ ID NO:1 by the deletion of 1-4 amino acids at the N-terminus of SEQ ID NO:1, based on the overall weight of all ungluconoylated and uncarbamoylated proteins in said composition that comprise at least 100 contiguous amino acids of the sequence of SEQ ID NO:1, and determined by LCMS after reductive dimethylation (stable isotope dimethyl labelling, SIDL) according to Tolonen et al. (2011). The composition shown in Table 15 comprising the +A variant (SEQ ID NO:1) shows 0.2% (w/w) of such protein deletions.

The composition of the invention preferably comprises less than 5% (w/w), less than 4% (w/w), less than 3% (w/w), less than 2% (w/w) and more preferably less than 1% (w/w) protein molecules that differ from the amino acid sequence of SEQ ID NO:2 by either (i) the deletion of 1-4 amino acids at the N-terminus of SEQ ID NO:2, or (ii) the addition of a single amino acid at the N-terminus of SEQ ID NO:2, based on the overall weight of all ungluconoylated and uncarbamoylated proteins in said composition that comprise at least 100 contiguous amino acids of the sequence of SEQ ID NO:2, and determined by LCMS after reductive dimethylation (stable isotope dimethyl labelling, SIDL) according to Tolonen et al. (2011). The composition shown in Table 15 comprising the +S variant (SEQ ID NO:2) shows 3.1% (w/w) of proteins according to (i) or (ii).

The composition of the invention preferably comprises less than 5% (w/w), less than 4% (w/w), less than 3% (w/w), less than 2% (w/w) and more preferably less than 1% (w/w) protein molecules that differ from the amino acid sequence of SEQ ID NO:2 by the deletion of 1-4 amino acids at the N-terminus of SEQ ID NO:2, based on the overall weight of all ungluconoylated and uncarbamoylated proteins in said composition that comprise at least 100 contiguous amino acids of the sequence of SEQ ID NO:2, and determined by LCMS after reductive dimethylation (stable isotope dimethyl labelling, SIDL) according to Tolonen et al. (2011). The composition shown in Table 15 comprising the +S variant (SEQ ID NO:2) shows 0.5% (w/w) of such protein deletions.

The composition of the invention preferably comprises more than 95% (w/w), more than 96% (w/w), more than 97% (w/w), more than 98% (w/w), and more preferably more than 99% (w/w) of protein molecules having a length of 143 amino acids and consisting of the amino acid sequence of SEQ ID NO:1, based on the overall weight of all ungluconoylated and uncarbamoylated proteins in said composition that comprise at least 100 contiguous amino acids of the sequence of SEQ ID NO:1, and determined by LCMS after reductive dimethylation (stable isotope dimethyl labelling, SIDL) according to Tolonen et al. (2011).

The composition of the invention preferably comprises more than 95% (w/w), more than 96% (w/w), more than 97% (w/w), more than 98% (w/w), and more preferably more than 99% (w/w) of protein molecules having a length of 143 amino acids and consisting of the amino acid sequence of SEQ ID NO:2, based on the overall weight of all ungluconoylated and uncarbamoylated proteins in said composition that comprise at least contiguous 100 amino acids of the sequence of SEQ ID NO:2, and determined by LCMS after reductive dimethylation (stable isotope dimethyl labelling, SIDL) according to Tolonen et al. (2011).

The composition of the invention preferably comprises more than 90% (w/w), more than 91% (w/w), more than 92% (w/w), more than 93% (w/w), more than 94% (w/w), and more preferably more than 95% (w/w) of protein molecules having a length of 143 amino acids and consisting of the amino acid sequence of SEQ ID NO:1, based on the overall weight of all proteins in said composition that comprise at least 100 contiguous amino acids of the sequence of SEQ ID NO:1, and measured by liquid chromatography mass spectrometry (LCMS), wherein gluconoylated proteins, carbamoylated proteins, dehydrated proteins and Na+ adducts are ignored for the calculation of the percentage.

The composition of the invention preferably comprises more than 90% (w/w), more than 91% (w/w), more than 92% (w/w), more than 93% (w/w), more than 94% (w/w), and more preferably more than 95% (w/w) of protein molecules having a length of 143 amino acids and consisting of the amino acid sequence of SEQ ID NO:2, based on the overall weight of all proteins in said composition that comprise at least 100 contiguous amino acids of the sequence of SEQ ID NO:2, and measured by liquid chromatography mass spectrometry (LCMS), wherein gluconoylated proteins, carbamoylated proteins, dehydrated proteins and Na+ adducts are ignored for the calculation of the percentage.

The proteins of the invention share at least one biological activity of the naturally occurring human mature MYDGF protein which renders them useful for being applied therapeutically. The invention hence also relates to a composition as described above for use as a medicament in particular for the uses known for MYDGF, see e.g. WO 2014/111458 and WO 2021/148411.

Preferably, the protein is active in (i) treating or preventing a disease or condition selected from the group consisting of injury, wounding, ischemia, reperfusion injury, trauma, mechanical overload, intoxication, surgery, primary or acquired cardiomyopathy, postischemic contractile dysfunction, myocardial infarction, preferably acute myocardial infarction, angina pectoris, heart failure, inflammation of the heart, heart insufficiency, hypertrophy, and fibrosis; (ii) promoting or improving heart tissue regeneration, cardiomyocyte proliferation, neovascularisation, heart function or left ventricular systolic function after myocardial infarction; (iii) protecting cardiomyocyte from death, e.g. through apoptosis or necrosis; or (iv) decreasing infarct size after myocardial infarction, preferably acute myocardial infarction. See WO 2014/111458.

The cardiomyopathy to be treated may be inherited cardiomyopathy or cardiomyopathy caused by spontaneous mutations. The cardiomyopathy to be treated may also be an acquired cardiomyopathy, preferably ischemic cardiomyopathy caused by atherosclerotic or other coronary artery diseases, cardiomyopathy caused by infection or intoxication of the myocardium, hypertensive heart disease caused by pulmonary arterial hypertension and/or arterial hypertension or diseases of the heart valves. The cardiomyopathy is preferably a cardiomyopathy selected from the group consisting of hypertrophic cardiomyopathy (HCM or HOCM), arrythmogenic right ventricular cardiomyopathy (ARVC), isolated ventricular non-compaction mitochondrial myopathy, dilated cardiomyopathy (DCM), restrictive cardiomyopathy (RCM), Takotsubo cardiomyopathy, Loeffler endocarditis, diabetic cardiomyopathy, alcoholic cardiomyopathy, or obesity-associated cardiomyopathy.

The heart failure to be treated preferably is chronic heart failure. The heart failure or chronic heart failure may be heart failure with preserved ejection fraction (HFpEF), heart failure with reduced ejection fraction (HFrEF), or heart failure with mid-range ejection fraction (HFmrEF). See WO 2021/148411.

It is particularly preferred that the protein described above has at least part of the activity of the naturally occurring human mature MYDGF protein in enhancing coronary artery endothelial cell or coronary artery endothelial cell proliferation. In particular, it is preferred that the protein has at least 50% of the activity of the naturally occurring human mature MYDGF protein in enhancing coronary artery endothelial cell proliferation, and preferably at least 60%, at least 70% at least 80% at least 90%, at least 95% or 100% of the said activity. The protein may also exert an activity in enhancing coronary artery endothelial cell proliferation which is higher than the activity of the naturally occurring human mature MYDGF protein, such as an activity of at least 110%, at least 120% at least 130% at least 90%, at least 140%, at least 150% at least 160% at least 180%, at least 190%, at least 200%, at least 250%, at least 300%, at least 350%, at least 400%, at least 450%, or at least 500% relative to the human mature MYDGF protein. Preferably, the activity in enhancing coronary artery endothelial cell proliferation is determined as described in the potency assay of Example 6 below. The activity of the protein is calculated by the formula: Activity [%]=EC₅₀ of mature MYDGF/EC₅₀ of test protein)×100.

In another embodiment, it is preferred that the protein has at least 50% of the activity of the [+G]-HEK variant in enhancing coronary artery endothelial cell proliferation, wherein the [+G]-HEK variant has been manufactured as described in Polten et al. (2019) and Ebenhoch et al. (2019). It is particularly preferred that the protein has at least 60%, at least 70% at least 80% at least 90%, at least 95% or 100% of the said activity. The protein may also exert an activity in enhancing coronary artery endothelial cell proliferation which is higher than the activity of the [+G]-HEK variant, such as an activity of at least 110%, at least 120% at least 130% at least 90%, at least 140%, at least 150% at least 160% at least 180%, at least 190%, at least 200%, at least 250%, at least 300%, at least 350%, at least 400%, at least 450%, or at least 500% relative to the [+G]-HEK variant. Preferably, the activity in enhancing coronary artery endothelial cell proliferation is determined as described in the potency assay of Example 6 below. The activity of the protein is calculated by the formula: Activity [%]=EC₅₀ of [+G]-HEK)/EC₅₀ of test protein)×100.

In yet another embodiment, it is preferred that the protein described above has at least 50% of the activity of the naturally occurring human mature MYDGF protein in enhancing cardiomyocyte proliferation, and preferably at least 60%, at least 70% at least 80% at least 90%, at least 95% or 100% of the said activity. The protein may also exert an activity in enhancing cardiomyocyte proliferation which is higher than the activity of the naturally occurring human mature MYDGF protein, such as an activity of at least 110%, at least 120% at least 130% at least 90%, at least 140%, at least 150% at least 160% at least 180%, at least 190%, at least 200%, at least 250%, at least 300%, at least 350%, at least 400%, at least 450%, or at least 500% relative to the human mature MYDGF protein. Preferably, the activity in enhancing cardiomyocyte proliferation is determined as described in the potency assay of Example 7 below. The activity of the protein is calculated by the formula: Activity [%]=EC₅₀ of mature MYDGF/EC₅₀ of test protein)×100.

In yet another embodiment, it is preferred that the protein has at least 50% of the activity of the [+G]-HEK variant in enhancing cardiomyocyte proliferation, wherein the [+G]-HEK variant has been manufactured as described in Polten et al. (2019) and Ebenhoch et al. (2019). It is particularly preferred that the protein has at least 60%, at least 70% at least 80% at least 90%, at least 95% or 100% of the said activity. The protein may also exert an activity in enhancing cardiomyocyte proliferation which is higher than the activity of the [+G]-HEK variant, such as an activity of at least 110%, at least 120% at least 130% at least 90%, at least 140%, at least 150% at least 160% at least 180%, at least 190%, at least 200%, at least 250%, at least 300%, at least 350%, at least 400%, at least 450%, or at least 500% relative to the [+G]-HEK variant.

Preferably, the activity in enhancing cardiomyocyte proliferation is determined as described in the potency assay of Example 7 below. The activity of the protein is calculated by the formula:

$$Activity [ \% ] = {EC}_{50} of [ + G ] - HEK ) / {EC}_{50} of test protein ) \times 100.$$

In yet another embodiment, it is preferred that the protein described above enhances coronary artery endothelial cell proliferation with an EC₅₀ of less than 100 ng/ml, when measured in the potency assay of Example 6 described below. Preferably, the protein enhances coronary artery endothelial cell proliferation with an EC₅₀ of less than 95 ng/ml, less than 90 ng/ml, less than 85 ng/ml, less than 80 ng/ml, less than 75 ng/ml, less than 70 ng/ml, less than 65 ng/ml, or less than 60 ng/ml, when measured in the potency assay of Example 6.

In yet another embodiment, it is preferred that the protein enhances cardiomyocyte proliferation with an EC₅₀ of less than 100 ng/ml when measured in the potency assay of Example 7 described below. Preferably, the protein enhances coronary artery endothelial cell proliferation with an EC₅₀ of less than 95 ng/ml, less than 90 ng/ml, less than 85 ng/ml, less than 80 ng/ml, less than 75 ng/ml, less than 70 ng/ml, less than 65 ng/ml, less than 60 ng/ml, less than 55 ng/ml, less than 50 ng/ml, less than 45 ng/ml, less than 40 ng/ml, less than 35 ng/ml, less than 30 ng/ml, or less than 25 ng/ml, when measured in the potency assay of Example 7.

The invention also relates to a nucleic acid encoding a protein which upon maturation results in a protein as described above, i.e. a protein having the sequence of SEQ ID NO:1 or SEQ ID NO:2. The nucleic acid can be DNA or RNA. It is however preferred that the nucleic acid is a DNA molecule.

The invention also relates to a vector or plasmid comprising a nucleic acid encoding a protein which upon maturation results in one of the MYDGF proteins of the invention. Preferably, the vector will be an expression vector that allows for the expression of a protein that matures into the protein of SEQ ID NO:1 or SEQ ID NO:2 in a prokaryotic or eukaryotic cell. It is particularly preferred that the vector is a prokaryotic expression vector, i.e. a vector that allows recombinant protein expression in a prokaryotic cell environment. Even more preferably, the vector is a bacterial expression vector, i.e. a vector that allows recombinant protein expression in a bacterial cell. The vector will preferably comprise an origin of replication, a promotor, a polylinker for cloning, a transcription terminator, and a gene that allows selection, such as a gene encoding a protein that confers antibiotic resistance. A vast number of expression vectors have been described for E. coli and other bacterial hosts. Examples for vectors suitable for protein expression in E. coli cells comprise, for example, the vectors of the pBluescript series, the pUC series, the pQE series or the pET series. The vector preferably comprises an inducible promoter system that is able to initiate expression upon addition of an inducer compound.

In a preferred embodiment, the vector that harbours the nucleic acid which encodes the MYDGF protein described above is a vector of the pET type. These vectors typically comprise an origin of replication, a T7 promoter which is specific to the T7 RNA polymerase, a lac operator for binding the lacI repressor protein, a polylinker for cloning the nucleic acid sequence encoding the protein to be expressed, a transcription termination sequence, an ampicillin or kanamycin resistance gene and a lacI gene which codes for the lac repressor protein. In the absence of isopropyl-β-D-thiogalactopyranoside (IPTG) or lactose, the repressor protein binds to the lac operator, thereby inhibiting the T7 promoter and blocking expression of the target protein. The binding of IPTG or lactose to the lac repressor protein leads to a conformational change which causes detachment of the protein from the operator and induction of expression from the T7 promoter. Suitable pET vectors for use in the methods of the present invention comprise, but are not limited to, pET21a(+), pET24a(+), pET28a(+), pET29a(+), pET30a(+), pET41a(+), pET44a(+), pET21b(+), pET24b(+), pET26b(+), pET28b(+), pET29b(+), pET30b(+), pET42b(+) and pET44b(+). Vectors which are based on the pET-26b(+) backbone are particularly preferred. Further examples of suitable vectors are described, e.g. in “Cloning Vectors” (Pouwels et al. (eds.) Elsevier, Amsterdam New York Oxford, 1985).

The expression vector may be transformed into the eukaryotic or prokaryotic host cell by any suitable method. For example, an expression vector for use in E. coli may be introduced into the host cell, e.g. by electroporation or by chemical methods, such as calcium phosphate-mediated transformation, as described in Maniatis et al. 1982, Molecular Cloning, A laboratory Manual, Cold Spring Harbor Laboratory.

The invention also relates to a host cell comprising a protein, a nucleic acid, or a vector as described above. The host cell may be a eukaryotic or prokaryotic cell, but it is particularly preferred that it is a prokaryotic host cell, such as a bacterial cell. While the type of bacterial cell is not particularly limited, it is preferred that the host cell is an Escherichia coli cell, such as an Escherichia coli cell BL21 cell.

The invention also relates to a composition described above, i.e. a composition comprising a protein described above, preferably a composition obtainable by a method of recombinant expression in a cell-based expression system, such as the methods described in more detail below, for the preparation of a pharmaceutical composition.

The invention also relates to a pharmaceutical composition which comprises the protein described above or a composition described above. The composition may comprise, apart from the protein, a pharmaceutically acceptable carrier and other excipients that are commonly used for the formulation of pharmaceutical compositions. Generally, the pharmaceutical composition may be formulated for different routes of administration. It is preferred that the composition of the invention is formulated for parental administration, e.g. by intravenous, intraarterial, intracoronary or intravenous administration. Compositions suitable for injection or infusion may include solutions or dispersions and powders for the extemporaneous preparation of such injectable solutions or dispersions. The composition for injection must be sterile and should be stable under the conditions of manufacturing and storage. Preferably, the compositions for injection or infusion also include a preservative, such as a chlorobutanol, phenol, ascorbic acid, thimerosal, and the like. For intravenous administration, suitable carriers may comprise physiological saline, bacteriostatic water, Cremophor EL™ (BASF) or phosphate-buffered saline (PBS). Sterile solutions for injection or infusion can be prepared by incorporating the MYDGF protein in the required amount in an appropriate solvent followed by filter sterilization.

Apart from MYDGF protein, the pharmaceutical composition of the invention may further comprise additional active agents that are effective in (i) treating or preventing a disease selected form the group consisting of injury, wounding, ischemia, reperfusion injury, trauma, mechanical overload, intoxication, surgery, primary or acquired cardiomyopathy, postischemic contractile dysfunction, myocardial infarction, preferably acute myocardial infarction, angina pectoris, heart failure, inflammation of the heart, heart insufficiency, hypertrophy, and fibrosis; (ii) promoting or improving heart tissue regeneration, cardiomyocyte proliferation, neovascularisation, heart function or left ventricular systolic function after myocardial infarction; (iii) protecting cardiomyocyte from death, e.g. through apoptosis or necrosis; or (iv) decreasing infarct size after myocardial infarction, preferably acute myocardial infarction in a subject in need thereof.

For example, the pharmaceutical composition may comprise one or more angiotensin-converting enzyme (ACE) inhibitors, such as benazepril, zofenopril, perindopril, trandolapril, captopril, enalapril, lisinopril, and ramipril. The pharmaceutical composition may also comprise one or more diuretics, including chlorothiazide, hydrochlorothiazide, bendroflumethiazide, spironolactone, chlorthalidone, methyclothiazide, polythiazide, triamterene, furosemide, ethacrynic acid, metolazone, bumetanide, indapamide, amiloride, acetazolamide, torsemide and eplerenone. The pharmaceutical composition may also comprise one or more beta blockers, including acebutolol, atenolol, betaxolol, bisoprolol, carvedilol, celiprolol, esmolol, metoprolol, nebivolol, propranolol, sotalol, and/or timolol.

If the protein is used in combination with any of the above additional active agents, e.g. with an ACE inhibitor, a diuretic and/or a beta blocker, the two active agents may also be administered separately from each other, i.e. in the form of separate pharmaceutical compositions, one containing the MYDGF protein, and the other containing the additional active agent. The separate compositions can be administered simultaneously, i.e. at the same time at two distinct sites of administration, or they may be administered sequentially (in either order) to the same site or to different sites of administration.

The invention also relates to a protein or a pharmaceutical composition as described above for use as a medicament. More specifically, the protein or pharmaceutical composition is suitable for being used a medicament for (i) treating or preventing a disease or condition selected from the group consisting of injury, wounding, ischemia, reperfusion injury, trauma, mechanical overload, intoxication, surgery, primary or acquired cardiomyopathy, postischemic contractile dysfunction, myocardial infarction, preferably acute myocardial infarction, angina pectoris, heart failure, inflammation of the heart, heart insufficiency, hypertrophy, and fibrosis; (ii) promoting or improving heart tissue regeneration, cardiomyocyte proliferation, neovascularisation, heart function or left ventricular systolic function after myocardial infarction; (iii) protecting cardiomyocyte from death, e.g. through apoptosis or necrosis; or (iv) decreasing infarct size after myocardial infarction, preferably acute myocardial infarction.

The invention also relates to a method for (i) treating or preventing a disease or condition selected from the group consisting of injury, wounding, ischemia, reperfusion injury, trauma, mechanical overload, intoxication, surgery, primary or acquired cardiomyopathy, postischemic contractile dysfunction, myocardial infarction, preferably acute myocardial infarction, angina pectoris, heart failure, inflammation of the heart, heart insufficiency, hypertrophy, and fibrosis; (ii) promoting or improving heart tissue regeneration, cardiomyocyte proliferation, neovascularisation, heart function or left ventricular systolic function after myocardial infarction; (iii) protecting cardiomyocyte from death, e.g. through apoptosis or necrosis; or (iv) decreasing infarct size after myocardial infarction, preferably acute myocardial infarction, in a subject in need thereof, wherein said method comprises the administration of an effective amount of a pharmaceutical composition as described above comprising the protein of SEQ ID NO:1 or SEQ ID NO:2.

The invention also provides a method for the recombinant expression of a MYDGF protein in a host cell, said method comprising the following steps:
(a) providing a host cell as described hereinabove, preferably a host cell that comprises a nucleic acid encoding a protein which after maturation consists of 143 amino acids having the amino acid sequence of SEQ ID NO:1 or SEQ ID NO:2;
(b) culturing the host cell under conditions that allow the expression of the MYDGF protein;
(c) isolating inclusion bodies containing the MYDGF protein from the host cell; and
(d) solubilising the inclusion bodies and refolding the MYDGF protein.

The above method of the invention is directed to the production of the proteins set forth in SEQ ID NO:1 and SEQ ID NO:2. The method makes use of a host cell as described hereinabove, preferably a prokaryotic host cell that comprises a nucleic acid encoding a protein which upon maturation results in one of the proteins of the invention. The bacterial host cell used in the method of the invention preferably contains a nucleic acid encoding a protein which upon maturation results in the protein of SEQ ID NO:1 or a nucleic acid encoding a protein which upon maturation results in the protein of SEQ ID NO:2 which is inserted in an expression vector that allows the expression of the recombinant protein in the host cell. The host cell can be any type of eukaryotic or prokaryotic cell that is suitable for being used for the expression of recombinant exogenous proteins, i.e. proteins that are not naturally produced by the host cell. Preferably, the host cell is a prokaryotic cell, such as a bacterial cell. More preferably the cell is a bacterial cell that belongs to the genus Escherichia, and even more preferably to the species E. coli. The use of E. coli strain BL21 or a derivative strain thereof is most preferred.

Preferably, step (a) of the above method comprises (i) providing a host cell that comprises a nucleic acid that contains an open reading frame, flanked by start and stop codon, according to the sequence of SEQ ID NO:11 or SEQ ID NO:12 operably linked to a promotor; or
(ii) providing a host cell that comprises a nucleic acid encoding a protein which before maturation consists of 144 amino acids having the amino acid sequence of SEQ ID NO: 15 or SEQ ID NO:16.

The amino acid sequences of SEQ ID NO:15 or SEQ:16 are MYDGF proteins which include the N-terminal methionine residue derived from the start codon. These proteins are subjected to a maturation process which removes the N-terminal methionine. The maturation preferably is effected by one or more host cell-derived aminopeptidases, more preferably one or more host cell-derived methionine aminopeptidases. For example, the one or more aminopeptidases may be produced by the bacterial host cell that is used for recombinant expression, e.g. E. coli. It is particularly preferred that step (a) comprises providing a host cell that comprises a nucleic acid of SEQ ID NO:7 or SEQ ID NO:8, which are expression vectors which are preferably circular, coiled or supercoiled.

In step (b) of the method, the bacterial host cell is cultured under conditions that allow for the expression of the protein in the bacterial host cell. The conditions that provide for the expression of the MYDGF protein will depend on the prokaryotic host cell and the expression vector used in the process. Suitable conditions can be readily selected and applied by a skilled person. For example, if an inducible bacterial expression system is used, such as a pET vector, the conditions that allow for the expression of the target protein will normally include a culturing temperature of between 20-42° C., preferably 30-40° C., and more preferably 35-38° C. The culture medium will typically have a pH of between 6.5 and 9.0, more typically 7.0 to 8.0, and preferably 7.5. Fermentation of the culture can be continued for a time period ranging from several hours to several days. The cells can be cultured in a batch or fed-batch process. For example, if the culturing is performed as a batch process, the culturing time normally ranges from about 12 hours to about 36 hours. When using a continuous process, fermentation times might be up to 21 days or longer. Suitable methods for the culturing of bacterial host cells are described in the Encyclopaedia of Bioprocess Technology: Fermentation, Biocatalysis, and Bioseparation, Volumes 1-5, Flickinger, M. C., Drew, S. W. (eds.), 1999 John Wiley & Sons. Preferably, the protein is expressed in the host cell using an inducible expression system, e.g. a system that allows initiating protein expression by the addition of an inducer compound like IPTG or lactose to the culture medium.

The proteins expressed in this way will accumulate in the host cell in insoluble form in so-called inclusion bodies. This means that the expressed proteins accumulate intracellularly and are deposited in the form of insoluble aggregates of inactive, misfolded proteins.

In step (c) of the method of the invention, the inclusion bodies containing the insoluble MYDGF protein are isolated from the host cell. For that purpose, the bacterial host cells are harvested after culturing and disrupted, e.g. by high-pressure homogenization or other commonly known procedures of cell lysis. Inclusion bodies can be isolated from the lysates by different methods, e.g. by tubular centrifugation, such as high speed tubular centrifugation. Methods for the isolation of inclusion bodies from bacterial cells are commonly known and described, for example, in Peternel & Komel (2010) and Eggenreich et al. (2020).

In step (d) of the method of the invention, the MYDGF protein in the isolated inclusion bodies obtained from step (c) is solubilised and refolded. Methods for solubilising proteins are known and include, for example, the incubation of inclusion bodies in the presence of urea, guanidine hydrochloride (GuHCl), and/or DTT, followed by filtration. It is preferred herein that the refolding of the proteins is performed in the presence of urea. Filtration may include one or more of depth filtration, ultrafiltration and/or diafiltration. Methods for refolding proteins are likewise known and include, for example, the incubation of proteins solubilized from inclusion bodies in the presence of urea, CaCl₂, and/or cystamine. Kits for solubilising and refolding proteins from inclusion bodies are marketed by different manufacturers.

The method may also comprise a step (e) in which a refolded MYDGF protein of 143 amino acids having the amino acid sequence of SEQ ID NO:1 or SEQ ID NO:2 is obtained. This step follows step (d) of the above method.

Preferably, the method of the invention comprises the additional step (f) in which the solubilized and refolded MYDGF protein obtained from step (e) is further purified. Protein purification can be conducted in accordance with routine methods and may include one or more of ultrafiltration, diafiltration, hydrophobic interaction chromatography and/or anion ion exchange chromatography.

Preferably, the purification in step (f) is effected by anion exchange chromatography or hydrophobic interaction chromatography. These types of chromatography may be performed by contacting the MYDGF protein to the chromatography resin material under conditions that allow for the adsorption of the MYDGF protein to the resin. The resin material is then optionally washed to remove impurities, such as non-proteinaceous material or proteins other than MYDGF. In a final step, the MYDGF protein is eluted from the resin.

More preferably, purification in step (f) is effected by anion exchange chromatography. If an anion exchange chromatography is used, the adsorption of the MYDGF protein to the anion exchange chromatography resin is preferably performed under conditions of low ionic strength, e.g. at a conductivity of less than 3 mS/cm, less than 2 mS/cm, less than 1.5 mS/cm or less than 1 mS/cm. Elution can be achieved by increasing the salt concentration and/or lowering the pH of the liquid phase, i.e. the mobile phase.

The method of the invention allows to produce the protein of SEQ ID NO:1 or SEQ ID NO:2 in particularly high amounts. As shown in Examples 2 and 3, the proteins can be produced with the method of the invention at a productivity of more than 0.4 g protein per 100 g cells, and preferably more than 0.5 g protein per 100 g cells, more than 0.6 g protein per 100 g cells, more than 0.7 g protein per 100 g cells, more than 0.8 g protein per 100 g cells, more than 0.9 g protein per 100 g cells, and more preferably more than 1.0 g protein per 100 g cells. Stated differently, the method of the invention allows to produce the protein of SEQ ID NO:1 or SEQ ID NO:2 in an amount of more than 100 g protein per batch, and preferably more than 150 g protein per batch, more than 200 g protein per batch, more than 250 g protein per batch, more than 300 g protein per batch, and more preferably more than 350 g or 400 g protein per batch (see Example 3).

The invention also relates to the use of a bacterial host cell as described elsewhere herein host cell for the recombinant expression of a MYDGF protein. The host cell is a prokaryotic or eukaryotic cell which comprises a nucleic acid, plasmid or vector that encodes a protein as described herein above.

A further embodiment of the invention is a protein according to one embodiment mentioned above which is obtainable by heterologous expression in bacteria, and preferably by a method described hereinabove. A further embodiment of the invention is a protein according to one embodiment mentioned above which is obtainable by production in the form of inclusion bodies and refolding. A further embodiment of the invention is a composition comprising a MYDGF protein, wherein said composition is obtainable by a heterologous expression in bacteria, and preferably by a method described hereinabove, wherein said composition comprises less than 1% (w/w) of protein molecules that are shorter than 143 amino acids.

In yet another aspect, the invention provides a method for producing a MYDGF protein in a cell-based expression system, comprising
(a) providing a host cell, preferably a host cell that comprises a nucleic acid encoding a protein which after maturation consists of SEQ ID NO:1 or SEQ ID NO:2;
(b) culturing the host cell under conditions that allow the expression of the protein;
(c) isolating inclusion bodies containing MYDGF protein from the host cell; and
(d) solubilising the inclusion bodies and refolding the MYDGF protein.

The distinct method steps of the method have been described hereinabove. It is once again preferred that the refolding of the proteins is performed in the presence of urea. Accordingly, the refolding of the protein in step (d) preferably comprises the incubation of the protein in the presence of urea.

The method may also comprise a step (e) in which a refolded MYDGF protein of 143 amino acids having the amino acid sequence of SEQ ID NO:1 or SEQ ID NO:2 is obtained. This step follows step (d) of the above method.

The above method may also comprise an additional step (f) in which the MYDGF protein expressed by the host cell and isolated from the inclusion bodies are purified. This step may comprise methods which are commonly used in the field of protein purification, such as ultrafiltration, diafiltration and/or anion ion exchange chromatography.

As described elsewhere herein, the host cell may be any host cell which is suitable for being used in the production of recombinant proteins. The host cell may be eukaryotic or prokaryotic.

The use of a prokaryotic host cell is preferred. It is even more preferred that the host cell is a bacterial cell, such as an Escherichia coli cell. The use of Escherichia coli cells of strain BL21 or a derivative strain thereof is particularly preferred.

The nucleic acid contained by the host cell which encodes a protein which upon maturation results in the protein of SEQ ID NO:1 or SEQ ID NO:2 may be a DNA or RNA molecule, and preferably a DNA molecule. The nucleic acid may be contained in a vector, such as a eukaryotic or prokaryotic expression vector. Preferably, the vector is a prokaryotic expression vector as described elsewhere herein.

The invention also relates to a composition obtainable from the above method, wherein said composition comprises a protein of 143 amino acids having the amino acid sequence of SEQ ID NO:1 or SEQ ID NO:2. The composition preferably comprises less than 1% (w/w) of protein molecules that are shorter than 143 amino acids, as measured by LCMS.

The composition will comprise no or only a small amount of carbamoylated protein. In a preferred embodiment, the composition comprises detectable amounts of carbamoylated proteins. For example, in one embodiment the amount of carbamoylated proteins is 0.01% (w/w), 0.02% (w/w), 0.05% (w/w), or 0.1% (w/w). At the same time, the overall amount of carbamoylated protein will be limited to less than 8% (w/w), and preferably less than 7% (w/w), less than 6% (w/w) or less than 5% (w/w) of the proteins in said composition. In another preferred embodiment, the composition comprises no detectable amounts of carbamoylated proteins.

Similarly, composition will comprise no or only a small amount of gluconoylated protein. In a preferred embodiment, the composition comprises detectable amounts of gluconoylated proteins. For example, in one embodiment the amount of gluconoylated proteins is 0.01% (w/w), 0.02% (w/w), 0.05% (w/w), or 0.1% (w/w). At the same time, the overall amount of gluconoylated protein will be limited to less than 6% (w/w), less than 5% (w/w), less than 4% (w/w) or less than 3% (w/w) of the proteins in said composition. In another preferred embodiment, the composition comprises no detectable amounts of gluconoylated proteins.

In a particularly preferred embodiment, the composition comprises detectable amounts of carbamoylated proteins, wherein however less than 7% (w/w) or less than 5% (w/w) of the proteins in the composition are carbamoylated. For example, the composition may comprise at least 0.05% (w/w) or at least 0.1% (w/w) carbamoylated proteins, wherein however less than 7% (w/w) of the proteins in the composition are carbamoylated. In yet another particularly preferred embodiment, the composition may comprise at least 0.05% (w/w) or at least 0.1% (w/w) carbamoylated proteins, wherein however less than 5% (w/w) of the proteins in the composition are carbamoylated.

Similarly, in another particularly preferred embodiment, the composition comprises detectable amounts of gluconoylated proteins, wherein however less than 5% (w/w) or less than 3% (w/w) of the proteins in the composition are gluconoylated. For example, the composition may comprise at least 0.05% (w/w) or at least 0.1% (w/w) gluconoylated proteins, wherein however less than 5% (w/w) of the proteins in the composition are gluconoylated. In yet another particularly preferred embodiment, the composition may comprise at least 0.05% (w/w) or at least 0.1% (w/w) gluconoylated proteins, wherein however less than 3% (w/w) of the proteins in the composition are gluconoylated.

In one embodiment, the composition comprises a protein having the amino acid sequence of SEQ ID NO:1 along with variants thereof which are shorter in length and exert 100% sequence identity over their entire length with the amino acid sequence of SEQ ID NO:1, wherein the length is at least 100 amino acids with no gaps being allowed in the alignment. Within said composition, the ratio of the signal obtained from the protein according to SEQ ID NO:1 and the sum of signals obtained from said shorter variants, as determined by LCMS after reductive dimethylation (stable isotope dimethyl labelling, SIDL) according to Tolonen et al (2011), is higher than 20, and preferably higher than 30, 40, 50, 60, 70, 75, 80, 85, 90, 100, 125, 150, 175, 200, 225, 250, 275, 300, 325, 350, 375, 400, 425, and more preferably higher than 450. As can be seen from Table 15 below, the ratio of the signal obtained from the protein according to SEQ ID NO:1 and the signals obtained from shorter variants was found to be 466, as determined by LCMS according to Tolonen et al (2011).

In one embodiment, the composition comprises a protein having the amino acid sequence of SEQ ID NO:2 along with variants thereof which are shorter in length and exert 100% sequence identity over their entire length with the amino acid sequence of SEQ ID NO:2, wherein the length is at least 100 amino acids with no gaps being allowed in the alignment. Within said composition, the ratio of the signal obtained from the protein according to SEQ ID NO:2 and the sum of signals obtained from said shorter variants, as determined by LCMS after reductive dimethylation (stable isotope dimethyl labelling, SIDL) according to Tolonen et al (2011), is higher than 20, and preferably higher than 50, 75, 100, 125, 150, and more preferably higher than 175 or 180. As can be seen from Table 15 below, the ratio of the signal obtained from the protein according to SEQ ID NO:2 and the signals obtained from shorter variants was found to be 186, as determined by LCMS according to Tolonen et al (2011).

Preferably, the composition comprises a protein which is folded such that more than 70%, and preferably more than 80%, more than 90%, or more than 95%, of the ¹H and/or ¹⁵N peaks in the two-dimensional nuclear magnetic resonance spectroscopy (2D-NMR) map result in combined chemical shift deviation (CCSD) values below 0.01 ppm when compared to the corresponding peaks in Table 1.

The above composition contains a minimum amount of impurities or post-translational modifications and can thus be used for the preparation of a pharmaceutical composition. The pharmaceutical composition may be prepared as described elsewhere herein.

The above composition or pharmaceutical composition is preferably for use as a medicament, and it may be applied as described above, e.g. for treating or preventing heart insufficiency, treating cardiomyopathy, promoting heart tissue regeneration, promoting cardiomyocyte proliferation, promoting neovascularisation, promoting heart function, decreasing infarct size, treating or preventing fibrosis, treating or preventing hypertrophy, or treating or preventing heart failure in a subject in need thereof.

### BRIEF DESCRIPTION OF THE FIGURES

FIG. 1 shows the NMR spectrum of the MYDGF [+A] variant described in Example 5.

FIG. 2 shows the NMR spectrum of the MYDGF [+G-HEK]variant described in Example 5.

FIG. 3 shows a superposition of both spectra according to FIGS. 1 and 2.

FIG. 4 shows dose-response curves for different MYDGF variants in the HCAEC migration assay. Recovery data were derived from cells that had been treated either with vehicle (control), VEGFA (50 ng/mL), different concentrations of [+G]-HEK (reference) or different concentrations of the MYDGF variant [+A] batch V301, respectively. 4-PL curve fits for [+G]-HEK and the respective [+A] batch are shown.

FIG. 5 shows dose-response curves for different MYDGF variants in the HCAEC migration assay. Recovery data were derived from cells that had been treated either with vehicle (control), VEGFA (50 ng/mL), different concentrations of [+G]-HEK (reference) or different concentrations of the MYDGF variant [+A] batch V302, respectively. 4-PL curve fits for [+G]-HEK and the respective batch [+A] are shown.

FIG. 6 shows dose-response curves for different MYDGF variants in the HCAEC migration assay. Recovery data were derived from cells that had been treated either with vehicle (control), VEGFA (50 ng/mL), different concentrations of [+G]-HEK (reference) or different concentrations of the MYDGF variant [+A] batch V303, respectively. 4-PL curve fits for [+G]-HEK and the respective [+A] batch are shown.

FIG. 7 shows a comparison of different MYDGF variants and batches in ischemia/reperfusion assays. The metabolic activity was determined for cells that had been stimulated with vehicle (control), IGF-1 (50 ng/mL), different concentrations of [+G]-HEK (reference) or different concentrations of MYDGF (batches V301, V302 and V303), respectively. 4-PL curve fits for [+G]-HEK and the respective batch [+A] are shown.

FIG. 8 shows the cardiac function in FVB/N mice as assessed by echocardiography. Left ventricular end-diastolic area (LVEDA), left ventricular end-systolic area (LVESA), and fractional area change (FAC) as assessed by transthoracic echocardiography on day 6 (A) and day 28 (B) after sham or I/R surgery in FVB/N mice. Treatments and animal numbers (within columns) are indicated. MYDGF designates the human protein, Mydgf the murine protein.

FIG. 9 shows (A) the infarct scar size on day 28 after MI in FVB/N mice. Example images and summary data are shown. Scar size was assessed by Masson's trichrome staining. Treatments and animal numbers (within columns) are indicated. MYDGF designates the human protein, Mydgf the murine protein. (B) Capillary density on day 28 after MI in FVB/N mice. Example images and summary of data are shown. Capillary density was assessed by fluorescent IB4/WGA staining. Treatments are indicated. 6 mice per group were used. MYDGF designates the human protein, Mydgf the murine protein.

### EXAMPLES

The invention will be illustrated by the following Examples which are given by way of example only. Specifically, the Examples describe the generation of the production strain and the heterologous expression and purification of MYDGF variants.

As described in the more detail in the below Examples, the production process for human MYDGF was first developed at 5 L scale using a Research Cell Bank (RCB), and then verified by consolidation runs at 20 L scale using a GMP-compliant working cell bank (WCB). Finally, expression was transferred to a current Good Manufacturing Practice (cGMP) facility in a 200 L scale which resulted in batch yields of 16-18 kg wet inclusion bodies (IBs). The downstream process for purification of the MYDGF protein from inclusion bodies was developed first at laboratory scale, then verified by consolidation runs at pilot scale using inclusion bodies from a 10 L fermentation aliquot and finally transferred to a cGMP facility where one downstream batch starts with 10 kg wet IBs representing a fermentation aliquot of about 110-125 L.

Non-clinical and clinical batches of MYDGF were manufactured at the 200 L scale. Several batches were performed in the GMP facility. The batches resulted in high yields of typically 330-355 g MYDGF from one 125 L fermentation aliquot. This reflects a yield of up to 2.84 g/L fermentation. The MYDGF produced by this process fulfilled all quality requirements necessary for the use in toxicological and clinical studies. Monomer content measured by HP size exclusion chromatography was routinely above 99% with high molecular weight impurities (aggregates) below 1% and low molecular weight impurities (fragments) below 0.1%. Endotoxin content was below 0.03 EU/mg protein. Host cell DNA content was <3 pg/mg protein.

### Example 1: Preparation of Vectors and Transfection

For the production of a cell bank, a derivative of Escherichia coli strain BL21 (DE3) was used that had been modified such that it does not produce phages. The strain was transformed with one of the vectors set forth in SEQ ID NO:7-10 carrying the gene encoding the respective MYDGF variant. The genes of the respective variants were codon-optimized for high expression rates in E. coli and synthesized by ATUM (Newark, California, USA). Plasmids encoding the following MYDGF variants were produced:
[+A] variant in which the N-terminal V residue in position +1 of the mature human MYDGF is preceded by an A residue (aa sequence set forth in SEQ ID NO:1: AVSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYS KAAFERESDVPLKTEEFEVTKTAVAHRPGAFKAELSKLVIVAKASRTEL). The expression vector encoding this variant is set forth in SEQ ID NO:7.
[+S] variant in which the N-terminal V residue in position +1 of the mature human MYDGF is preceded by an S residue (aa sequence set forth in SEQ ID NO:2: SVSEPTTVAFDVRPGGVVHSFSHNVGPGDKYTCMFTYASQGGTNEQWQMSLGTSEDHQHF TCTIWRPQGKSYLYFTQFKAEVRGAEIEYAMAYS KAAFERESDVPLKTEEFEVTKTAVAHRPGAFKAELSKLVIVAKASRTEL). The expression vector encoding this variant is set forth in SEQ ID NO:8).
[+G] variant in which the N-terminal V residue in position +1 of the mature human MYDGF is preceded by an G residue (aa sequence set forth in SEQ ID NO:3: GVSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYS KAAFERESDVPLKTEEFEVTKTAVAHRPGAFKAELSKLVIVAKASRTEL. The expression vector encoding this variant is set forth in SEQ ID NO:9.
[−V] variant in which the N-terminal V residue in position +1 of the mature human MYDGF is lacking (aa sequence set forth in SEQ ID NO:4: SEPTTVAFDVRPGGVVHSFSHNVGPGDKYTCMFTYASQGGT-NEQWQMSLGTSEDHQHFTCTIWRPQGKSYLYFTQFKAEVRGAEIEYAMAYSKAAFERESDVPLKTEEFEVTKT AVAHRPGAFKAELSKLVIVAKASRTEL. The expression vector encoding this variant is set forth in SEQ ID NO:10.

In case of a discrepancy between any of the sequences listed above and the sequences set forth in the attached sequence listing, the above paragraph will prevail. Hyphens that eventually show up within the sequence are a result of truncation due to text processing and must be ignored.

To prepare the expression strain, E. coli cells were transformed with the above described vector plasmids by electroporation using a Gene Pulser Xcell™ Electroporation System (BioRad). The protein was expressed in E. coli cell in the form of inclusion bodies (IBs) that accumulated in the cytoplasm, as further described in the below Examples.

### Example 2: Expression of MYDGF Variants

The expression and purification of the MYDGF variants was performed by the following general scheme:
1. Fermentation
2. IB Preparation
3. Solubilization/Refolding
4. Ultrafiltration/Diafiltration
5. Anion exchange chromatography
6. Preferably: Hydrophobic Interaction Chromatography
7. Preferably: Concentration and Formulation

### Example 2.1: Fermentation

One cell bank vial of the production strain obtained from Example 1 was thawed at room temperature. A pre-culture (PC) consisted of two 1 L shake flasks with 300 mL seed culture medium per flask. The composition of the seed medium is depicted in below Table 2. All buffer and media were prepared with reverse osmosis (RO) water and sterilized before use using either nanofiltration devices or heat-sterilization.

TABLE 2
Seed Culture Medium

| | Amount | |
|---|---|---|
| Component | per liter | Unit |

| Solution A (Basic Medium) | | | |
|---|---|---|---|
| 1 | Potassium Dihydrogen Phosphate | 3 | g |
| 2 | Di-Potassium Hydrogen Phosphate | 4.58 | g |
| 3 | Bacto Yeast Extract | 0.5 | g |
| 4 | Sodium Citrate × 2H₂O | 1 | g |
| 5 | Trace Element Solution | 0.2 | mL |
| | | 0.22 | g |
| 6 | Magnesium Sulfate × 7 H₂O | 0.4 | g |
| 7 | Ammonium Sulfate | 1.8 | g |
| 8 | Ammonium Chloride | 1.48 | g |
| 9 | RO-Water, Ad | 900 | mL |
| | | 906.93 | g |
| 10 | Antifoam (if needed) | | mL |
| Solution B (Sterile Additions) | | | |
| 11 | Calcium Chloride 2H₂O | 0.08 | g |
| 12 | Glucose anhydrous | 10 | g |
| 13 | RO-Water, ad | 100 | mL |
| | | 103.74 | g |
| 14 | Antibiotic (if needed) | | mL |

Antifoam and antibiotic were added as needed. Kanamycin was added as an antibiotic (to a final concentration of 50 g/mL). Each shake flask was inoculated with 100 μL of the production strain. The cultures were grown for approximately 9.4 h, aiming for an GD (optical density at 550 nm) of 1.75±0.5.

The main culture (MC) was performed in a 20 L gross stainless-steel bioreactor that contained 10 L batch medium. The composition of the batch medium is depicted in below Table 3.

TABLE 3
Batch Medium

| | Amount | |
|---|---|---|
| Component | per liter | Unit |

| Solution A (Basic Medium) | | | |
|---|---|---|---|
| 1 | Potassium Dihydrogen Phosphate | 3 | g |
| 2 | Di-Potassium Hydrogen Phosphate | 4.58 | g |
| 3 | Sodium Citrate × 2 H₂O | 1 | g |
| 4 | Trace Element Solution | 0.2 | mL |
| | | 0.22 | g |
| 5 | Magnesium Sulfate × 7 H₂O | 0.4 | g |
| 6 | Ammonium Sulfate | 1.8 | g |
| 7 | Ammonium Chloride | 1.48 | g |
| 8 | RO-Water, ad. | 900 | mL |
| | | 906.93 | g |
| 9 | Polypropylene glycol 2000 | 1 | mL |
| | | 1 | g |
| Solution B (Sterile Additions) | | | |
| 10 | Calcium Chloride × 2 H₂O | 0.08 | g |
| 11 | Glucose anhydrous | 10 | g |
| 12 | RO-Water, ad. | 100 | mL |
| | | 103.74 | g |

The batch medium was inoculated with 100 mL cell broth from the pre-culture. In the batch phase and exponential feed phase, fermentation process parameters were held constant at 33.5° C., pH 6.8, 1.0 bar head pressure and a DO set-point of 20%. An exponential feed (concentration 600 g/L glucose; μ=0.25 h⁻¹) was started after carbon source depletion was observed via a dissolved oxygen (DO) peak. The composition of the feed medium is depicted in below Table 4.

TABLE 4
Feed Medium

| | Amount | |
|---|---|---|
| Component | per liter | Unit |

| Solution A (Basic Medium) | | | |
|---|---|---|---|
| 1 | Glucose anhydrous | 600 | g |
| 2 | Calcium Chloride × 2 H₂O | 5.28 | g |
| 3 | RO-Water, ad. | 748.99 | mL |
| | | 962 | g |
| Solution B (Sterile Additions) | | | |
| 4 | Potassium Dihydrogen Phosphate | 7.8 | g |
| 5 | Di-Potassium Hydrogen Phosphate | 11.9 | g |
| 6 | Sodium Citrate × 2 H₂O | 66 | g |
| 7 | Magnesium Sulfate × 7 H₂O | 26.4 | g |
| 8 | Trace Element Solution | 13.2 | mL |
| | | 14.65 | g |
| 9 | RO-Water, ad. | 238.88 | mL |
| | | 292.03 | g |
| 10 | NaOH 10% | 14.4 | mL |
| | | 15.97 | g |

After 9 h of exponential feed rate (60.48 to 573.78 g/h), the feed rate was held constant at a rate of 573.78 g/h for the rest of the fermentation (11.5 h). A 60 min temperature ramp (from 33.5° C. to 30.0° C.) was initiated 11.5 h after the start of the exponential feed, and the ramps were completed directly before induction with IPTG was carried out. The culture was induced via a bolus of IPTG, 12.5 h after feed start. The MC was terminated 20.5 h after feed start. At the end of the culturing process, the culture broth was immediately cooled to <12° C., diluted with reverse osmosis (RO) water to a target wet cell weight (WCW) of 15% and bacterial cell mass was separated from the supernatant via centrifugation with a CEPA centrifuge. Biomass was harvested and, together with the supernatant, and transferred to downstream processing.

Product quantification was performed by using the LabChip GXII® system (Perkin Elmer) which provides is an automated high-throughput alternative to traditional SDS-PAGE and protein quantification. Sample preparation was performed with a liquid handling system (Tecan Freedom EVO 150). For product quantification from fermentation samples, analytical cell disruption of samples from fermentations was facilitated via enzymatic cell lysis. 90 μL of fermentation suspension was diluted in a 9:10 ratio (v/v) with cell disruption buffer (Lysonase™ (Merck) in FastBreak™ cell lysis reagent (Promega) with 32 μL Lysonase per 1 mL FastBreak™ reagent). For the total product determination (soluble and insoluble fraction), the samples were mixed before every pipetting step. Finally, the samples were diluted into the specific sample buffer of the system. To minimize the amount of required sample buffer, all dilution steps were carried out in PBS or another formulation buffer.

For the final dilution, 8 μL sample (from the PBS dilutions) or the standard curve samples were diluted in 28 μL non-reducing sample buffer in a 96-well plate (Eppendorf twin.tec PCR plate 95100401). For reducing conditions, 28 μL reducing sample buffer (with 35 mM DTT) was used. The plate was sealed with foil (Eppendorf PCR foil 0030127790), briefly centrifuged (30 s at 25 g) and denatured for 10 min at 70° C. After denaturation, the plate was centrifuged for 5 min at 2200 g to spin down any evaporated liquid. After centrifugation, the foil was removed and diluted via 140 μL DI water. The 96-well plate was sealed with a foil (Eppendorf twin.tec PCR plate 95100401) and the plate was centrifuged for 10 min at 2200 g to sediment any potential aggregates that would cause a malfunction of the LabChip analysis. After centrifugation, the plate was analyzed in the LabChip GXII with the setting “HT Protein Express 100 High Sensitivity”. The LabChip preparation was carried out according to the manufacturing guide. The standard curve was prepared by diluting reference material. As reference material, the [+G] variant was used that had been produced in HEK 293-6E cells as described in Polten (2019), see page 1303, 1′ column and Figure S1, and Ebenhoch (2019), see page 8 col. 1.

The quantification was carried out in the range from 1 mg/mL to 0.1 mg/mL via a linear fit. Reducing and non-reducing conditions did not change the integral area for quantification, however a shift in the running time was observed that did not influence the quantification.

### Example 2.2: Purification and Analysis of Inclusion Bodies

Frozen E. coli biomass obtained from Example 2.1 was resuspended 1:5 (w/v) with IB prep buffer 1 (1 M Urea, 50 mM Tris, 0.1% (v/v) Polysorbat 20, pH 7.5). Following 15 min resuspension with an ultraturrax, E. coli cells were disrupted by high-pressure homogenization using 3 passes at 650-700 bar. Dense and heavy inclusion bodies (IBs) as well as large cell fragments were separated by high speed tubular centrifugation using a GLE rotor (CEPA). The feed flow rate was 55 mL/min and the centrifugation speed 24,500 g. The tubing had an inner diameter of 3.2 mm. The recovered pellet was washed twice with HQ water. In all cases the pellets were diluted 1:5 (w/v) and re-suspended using an ultraturrax. After the HQ water steps the pellet mostly contains IBs.

### Example 2.3: Protein Solubilisation, Refolding and Purification

Frozen IBs obtained from Example 2.3 were solubilized at room temperature in solubilization buffer (8 M Urea, 0.14 M GuHCl, 6 mM DTT, 50 mM Tris, pH 8). The mixture was first stirred with an ultraturrax for 10 minutes and then with a propeller mixer for 180 minutes. The target concentration during solubilization was 5 mg/mL with a target volume of 100 mL. Subsequently the solubilization pool was filtered over a CUNO depth filter (filter E16E01A90ZB08A, 0.1 0.6 μm, 3M Deutschland GmbH, Neuss, Germany). Filters were pre equilibrated with water for injection (WFI) and solubilization buffer. Subsequently, the solubilization pool was loaded directly. The filtrate was collected by UV monitoring using an AKTA system. The inclusion body solubilisate was diluted with 1:5 refold buffer (4 M Urea, 0.3125 M Tris, 12.5 mM CaCl₂, 3.75 mM cystamine, pH 7). The recovered refold pool was stirred overnight. On the next day it was filtered over a CUNO depth filter (filter E16E01A60ZB05A, 3M Deutschland GmbH, Neuss, Germany).

After filtration with the depth filter, the filtrate was subjected to ultrafiltration/diafiltration (UFDF) to perform a buffer exchange. The UFDF was carried out with a Pellicon 3 membrane (88 cm², Ultracell 3 kDa, screen type C) using a diafiltration buffer (20 mM Tris, pH 9). A concentration factor of 2 and a diafiltration factor of 5 were used.

Following UFDF, the filtrate was subjected to ion exchange chromatography (IEX). A YMC Biopro IEX 75 μm column was used with a column diameter 1 cm, a bed height of 9 cm, and a column volume of 7.5 ml. The column was first equilibrated for 5 min with 3 column volumes (CV) equilibration buffer 1 (20 mM Hepes, 1 M NaCl pH 7) and subsequently for 5 min with 5 CV equilibration buffer 2 (20 mM Tris pH 9). After loading the filtrate, the filtrate was washed for 5 min with 5 CV 20 mM Tris pH 9. Protein was eluted for 5 min with 5 CV elution buffer 1 (20 mM Hepes, 1 M NaCl, pH 7) followed by 5 min with 10 CV elution buffer 2 (20 mM Hepes, pH 7) using a gradient (0% to 100% 20 mM Hepes, 1 M NaCl pH 7. Finally, the column was stripped for 5 min with 5 CV with 1 M HCl.

### Example 2.4: Protein Yields and Product Homogeneity

Purification was performed with all 4 variants at least once. In addition, a second purification experiment was performed with [+A] and [+S] variants. The Purification resulted in high yield and high purity for each of the four variants. In particular, the overall process yield after purification and refold for the [+A] variant was found to be 2.4 g/L for the first batch and 5.3 g/L for the second batch.

Analytical high performance size exclusion chromatography was performed in order to test for purity. The purified [+A] variant displayed a high purity of 99.75% main peak, 0.25% low molecular weight impurities and 0.0% aggregate levels. Similarly high purity levels were achieved with the [−V]variant (99.64% main peak, 0.0% low molecular weight impurities, 0.04% aggregates) and the [+S] variant (99.73% main peak, 0.2% low molecular weight impurities, 0.05% aggregates). In contrast, the [+G] variant product was less homogeneous when examined with high performance size exclusion chromatography showing 60.36% main peak purity and 39.64% aggregates.

Process yields from lab scale purification runs for all N-terminal variants are summarized in the following table:

TABLE 5a
Summary of lab scale production of MYDGF N-
terminal variants. Amount of MYDGF at different
process steps are provided in mg MYDGF

| | | MYDGF variant | | | |
|---|---|---|---|---|---|
| | | [+A] | [+G] | [−V] | [+S] |
| | | variant | variant | variant | variant |
| End of | optical density | 326 | 332 | 348 | 331 |
| fermentation | (OD 550 nm) | | | | |
| cell density | | | | | |
| End of | (amount wet | 330.17 | 310.42 | 337.58 | 311.65 |
| fermentation | cells g per L | | | | |
| wet cell | fermentation | | | | |
| weight | volume) | | | | |
| End of | (amount | 27.1 | 23.1 | 26.8 | 24.2 |
| fermentation | MYDGF: mg | | | | |
| product titer | per L | | | | |
| | fermentation | | | | |
| | volume) | | | | |

The fed batch fermentation process applied for all four variants resulted in very high cell densities at end of fermentation (GD at 550 nm of 326-348; wet cell mass of 310,42-337,58 g(L). Very high volumetric titers for recombinant MYDGF variants were achieved (23,1-27.1 g/L fermentation).

Zhao et al. (2020) have reported fermentation of a MYDGF-6His fusion protein in E. coli followed by extraction and purification from E. coli cell lysate.

TABLE 5b
Characteristics of the process according to Zhao et al. (2020)
compared to the process according to the invention.

| | Zhao et al. 2019 | Example 2 | Example 3 | Comments |
|---|---|---|---|---|
| overall | 216 mg purified (+Met)- | 788 mg purified | 825-888 mg | Process of the invention |
| productivity | MYDGF-6His per 100 g wet | (+Ala)-MYDGF per | purified (+Ala)- | provides a 4-fold higher |
| (related to | cells | 100 g wet cells | MYDGF bulk | productivity related to |
| amount of | | | drug substance | cell mass. This |
| cell mass) | | | per 100 g wet | productivity was |
| | | | cells. | shown to be scalable. |
| Overall | Unknown. However, based | Overall productivity | Overall productivity | Process of the invention |
| productivity | on the assumption of maximum | of 2.6 g purified | of 2.6-2.8 g | assumed to be >100-fold |
| (related to | 10 g cell mass per Liter | (+Ala)-MYDGF per | purified and | more productive when |
| fermentation | fermentation volume, | Liter Fermentation. | formulated | compared to |
| volume) | one would assume a | | (+Ala)-MYDGF | Zhao Process |
| | productivity of maximum | | per Liter | |
| | 21.6 mg (+Met)-MYDGF- | | Fermentation. | |
| | 6His per Liter fermentation | | | |
| | broth | | | |

TABLE 5c
Summary of the purification of rhMYDGF as disclosed in Zhao et al. (2020)

| cf.: table | Wet | | Protein | Total | | Yield (mg/ |
|---|---|---|---|---|---|---|
| S2 in Zhao | cells | Volume | concentration | protein | Purity | 100 g wet |
| et al. (2020) | (g) | (ml) | (mg/ml) | (mg) | (%) | bacteria) |
| Protein | 100 | 1000 | 0.82 | 820 | 46% | 377.2 |
| solubilization | | | | | | |
| Nickel-chelating | | 328 | 0.93 | 305.04 | 80% | 244.032 |
| column | | | | | | |
| Gel filtration | | 150 | 1.5 | 225 | 96%* | 216 |
| chromatography | | | | | | |
| column | | | | | | |

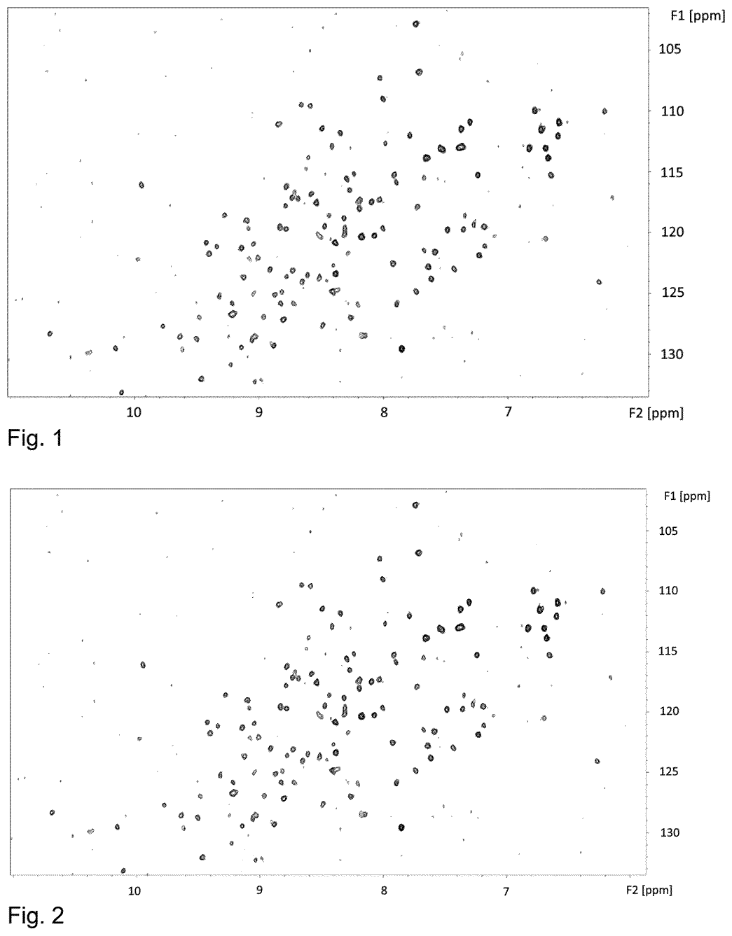

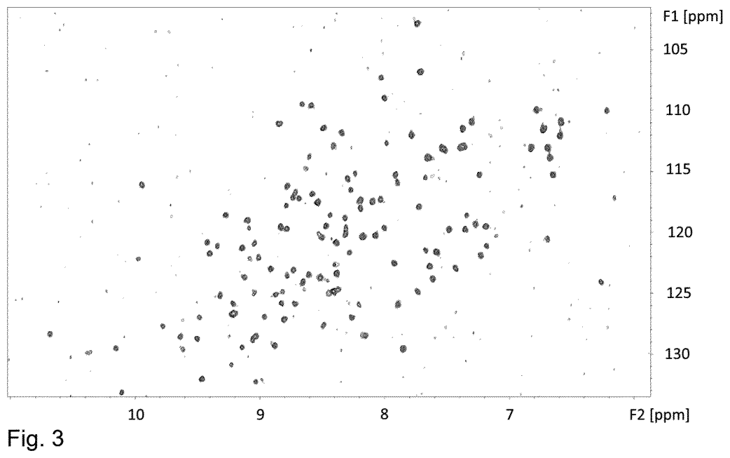

*See Zhao et al. (2020), at page 1192, section 3.1, lines 18-20 who refers to a purity of more than 95% with reference to FIG. 1C. See also FIG. 2A.

The above data was the average of three independent experiments. The protein was quantified by BCA method. The amount of target proteins was estimated by densitometry analysis of the protein band in SDS-PAGE gels. Total protein=protein concentration (mg/mL)×volume (mL). Yield=total protein (mg)×purity (%).

### Example 3: Advanced MYDGF Manufacturing Process

Based on Example 2 the manufacturing process was further developed for the [+A] variant. The fermentation process for MYDGF was first developed at 5 L scale using the Research Cell Bank (RCB), then verified by consolidation runs at 20 L scale using GMP working cell bank (WCB) and finally transferred to 200 L scale. A typical 200 L fermentation batch yields 16-18 kg wet IBs.

The downstream process for purification of MYDGF drug substance from intracellular inclusion bodies was developed first at laboratory scale, then verified by consolidation runs at pilot scale using inclusion bodies from a 10 L fermentation aliquot and finally transferred to a cGMP facility where one downstream batch starts with 10 kg wet IBs representing a fermentation aliquot of about 110-125 L.

TABLE 6a
Description of the CMC1a drug substance manufacturing process (upstream process part)

| | | Primary function |
|---|---|---|
| Process step | Description of the process step | of the process step |
| Cell Bank | Manufacturing of Master | |
| | Cell Bank (MCB) | |
| | and Working Cell Bank | |
| | (WCB). MCB is | |
| | derived from Research Cell | |
| | Bank (RCB). | |
| Seed Culture 1 | Seed culture (SC) is performed in shake | generate sufficient inoculum |
| | flasks containing 310 mL sterilized medium. | for main culture |
| | Sterilized seed culture medium is inoculated | |
| | with 100 μl of WCB. The seed culture is then | |
| | cultivated and terminated based on the | |
| | OD550 in one OD reference flask. | |
| | The SC is transferred to main culture. | |
| Main Culture | The main culture is conducted as a fed-batch | expand seed culture and |
| | fermentation. | expression of target protein |
| | Product formation is induced by a bolus | |
| | addition of an IPTG-solution. | |
| Biomass Harvest | Fermentation broth is cooled down and | recovery and concentration |
| | biomass is harvested through centrifugation | of host cells |
| | using a tubular bowl centrifuge. | |
| Resuspension of | The biomass is resuspended and diluted with | Resuspension of harvested |
| Biomass | a resuspension buffer. | biomass |
| Homogenization | The resuspended biomass is homogenized in | Disruption of E. coli cells |
| | three passes a high-pressure homogenizer at | |
| | 650 bar. The lysate is collected in a chilled | |
| | mobile tank. | |
| Harvest of IBs 1 | Harvest of Inclusion Bodies | Harvesting inclusion bodies |
| | (=Centrifugation of Homogenate) is | from homogenate |
| | performed with a tubular bowl centrifuge. | |
| IB Wash 1 | IBs 1 are collected after centrifugation of | Washing inclusion bodies 1 |
| | homogenate and diluted 1:5 (w/v) in purified | to remove cell debris and |
| | water. | impurities |
| Harvest of IBs 2 | Harvest of Inclusion Bodies (=Centrifugation | Harvesting inclusion bodies |
| | of Homogenate) is performed with a tubular | 2 from inclusion bodies 1 |
| | bowl centrifuge. | |
| IB Wash 2 | IBs 1 are collected after centrifugation of | Washing inclusion bodies 2 |
| | homogenate and diluted 1:5 (w/v) in purified | to remove cell debris and |
| | water. | impurities |
| Harvest of final | Harvest of Inclusion Bodies (=Centrifugation | Harvest of final |
| Inclusion Bodies | of Homogenate) is performed with a tubular | inclusion bodies |
| | bowl centrifuge. | |
| Packaging of final | The final IB pellet is packed into 500 g IB | Packaging of final Inclusion |
| IBs | aliquots and stored at −20° C. until further | Bodies into disposable |
| | processing. | containers |

TABLE 6b
Description of the CMC1a drug substance manufacturing process (downstream part)

| | | Primary function |
|---|---|---|
| Process step | Description of the process step | of the process step |
| Solubilization | Frozen Inclusion bodies are solubilized at 32 | Solubilization of frozen inclusion |
| | g IB/L target concentration using a buffer | bodies |
| | containing high chaotrope concentration and | |
| | reducing conditions. | |
| Depth Filtration 1 | The IB solution is filtered through a depth | Clarification of the IB solution |
| | filter. | Reduce particles |
| Refolding | The solubilized and filtered IB solution is | Formation of native like |
| | refolded by adding refold buffer in a ratio of | protein +A MYDGF variant |
| | 1:4 and subsequent incubation under constant | |
| | stirring. | |
| UFDF1 | The refold is first concentrated with a factor | Reduction of the process volume |
| | of 1.5, followed by continuous diafiltration | Removal chaotropes and refolding |
| | with at least 5 diavolumes. | additives. |
| | | Decrease conductivity |
| Depth Filtration 2 | The UFDF1 pool is filtered through a depth | Clarification of the IB solution |
| | filter. | Depletion of host related impurities |
| | | and product aggregates |
| AEX chromatography | The filtered UFDF1 pool is loaded onto an | Volume reduction |
| | anion exchange chromatography column. | Depletion of process related and |
| | Unbound impurities are removed by washing | product related impurities |
| | with low salt buffer. | |
| | The product is recovered by gradient elution. | |
| HIC chromatography | The AEX pool is first diluted with buffer con- | Depletion of process related and |
| | taining high salt concentration. The resulting | product related impurities |
| | HIC load is then loaded onto a hydrophobic | |
| | interaction chromatography column. | |
| | Unbound impurities are removed by washing | |
| | with high salt buffer. | |
| | The product is recovered by gradient elution. | |
| UFDF 2 | The HIC pool is first concentrated to a target | Concentration of the product |
| | concentration of ~20 g/L using a membrane | Depletion of process related |
| | with a nominal cut off of 5 kDa. | impurities of low molecular weight. |
| | Then the retentate is diafiltered with a sodium | |
| | chloride solution (5 diavolumes) followed by | |
| | 6 diavolumes of 20 mM Tris-HCl, pH 8.5. | |
| | The retentate is then concentrated to a con- | |
| | centration >70 g/L and captured from the | |
| | system. | |
| Formulation | 5-fold formulation buffer is added to the | Adjust excipient concentration |
| | UFDF 2 diafiltrate 2. | and |
| | | reach 50 ± 5 g/L, pH 8.5 ± 0.2 |
| DS filtration and | Filtration using a pre-sterilized | Bioburden removal |
| aliquotation | manifold equipped with | Packaging and storage |
| | a 0.2 μm filter; the bulk drug substance | |
| | is filtered into a bag for | |
| | homogenization; | |
| | aliquotation of DS into 6 L bags; | |
| | DS is frozen at <−60° C. to ≥−80° C. | |
| | stored at −40 ± 5° C. | |

Several batches were performed under GMP conditions. The batches resulted in high yields of typically 330-355 g MYDGF from one 125 L fermentation aliquot. This reflects an overall process yield of up to 2.84 g/L fermentation. The MYDGF drug substance produced by this process fulfilled all quality requirements necessary for the use in toxicological and clinical studies.

Monomer content measured by HP size exclusion chromatography was routinely above 99% with high molecular weight impurities (aggregates) below 1% and low molecular weight impurities (fragments) below 0.1%. Endotoxin content was below 0.03 EU per mg of the MYDGF protein. Host cell DNA content was <3 pg/mg protein.

### Example 4: Molecular Weight Analysis by LCMS and Advanced Molecular Weight Analysis by LCMS after Chemical Modification According to Tolonen et al. (“aLCMS”)

Samples of the folded and purified product obtained from Example 2 were subjected to Liquid Chromatography Mass Spectrometry (LCMS) analysis. Liquid Chromatography/Electrospray ionization Mass Spectrometry (LC-ESI-MS) was used to perform intact (non-reduced) molecular weight analysis on MYGDF constructs to (1) verify the sequence via conformity of the observed molecular weight to the predicted values for each sequence, and (2) capture a global profile of the net post-translational modifications (PTMs) on each protein. An Agilent 1290 UPLC with a 1.0 mm by 30 mm C3 POROS reversed phase column was used to desalt and introduce samples (0.5 pg/injection) into the mass spectrometer. A three minute binary gradient consisting of mobile phase A (98.9% water, 1% acetonitrile, 0.1% formic acid, and 2 mM ammonium acetate) and mobile phase B (70% isopropanol, 20% acetonitrile, 9.9% water, and 0.1% formic acid) that increased from 5% to 80% of mobile phase B at 150 μl/min was used to trap, desalt, and elute the protein from the column. Mass spectral data of the eluted material were acquired using an Agilent 6224 Time-of-Flight (TOF) MS, which was then processed (deconvoluted) using the maximum entropy algorithm within the Mass Hunter analysis software (Agilent). Data obtained by this method is referred to herein as “intact MW LCMS data” or data “measured by liquid chromatography mass spectrometry (LCMS)”.

For peptide-level sequence confirmation and site-specific post-transcriptional modification (PTM) analysis, aliquots of each sample were digested using trypsin and chymotrypsin separately to achieve complete sequence coverage. 100 pg of each sample was desalted and concentrated via acetone precipitation and centrifugal pelleting of the precipitated material. Each protein pellet was re-solubilized, denatured, and reduced in 10 μl of denaturation/reduction buffer (5% w/v sodium deoxycholate (SDC), 10 mM dithiothreitol (DTT), 20 mM ammonium bicarbonate) and incubated at 70° C. for 2 minutes, followed by a ten-fold dilution with 20 mM ammonium bicarbonate and 2 mM methionine. The reduced/denatured molecules were then split into two vials (50 pg each), whereupon trypsin and chymotrypsin were added separately at a 1:10 enzyme-to-substrate ratio to each tube, and the samples incubated for 10 minutes at 37° C. The reaction was quenched with addition of 10% v/v trifluoroacetic acid, resulting in 1% v/v final concentration of that reagent. The short (10 min) digestion step obviated the need for an alkylation step which is commonly used in peptide mapping. Precipitated sodium deoxycholate was removed by centrifugation at 16,000×g, and the peptide-containing supernatant was recovered and transferred to autosampler vials which were immediately stored at −80° C. until analysis. Data obtained by this method is referred to herein as “peptide mapping LCMS data”.

aLCMS: Additionally, as the first four N-terminal residues of the various MYGDF constructs have been shown to undergo fragmentation during electrospray (both at the intact and peptide levels), reductive dimethylation (also known as stable isotope dimethyl labeling (SIDL)) was performed according to Tolonen et al. (2019), on aliquots of the peptide digests to delineate between sample-derived versus electrospray-derived N-terminal truncations. Data obtained by this method is referred to herein as “LCMS after reductive dimethylation (stable isotope dimethyl labelling, SIDL)”. Briefly, 50 pg of peptides from each digest were immobilized into separate Waters Oasis SPE cartridges using a vacuum manifold. The SPE media and peptides were conditioned to pH 5.5 with citrate buffer (90 mM citric acid, 230 mM divalent sodium phosphate), and 10 ml of 0.8% v/v formaldehyde (in citrate buffer) and 120 mM sodium cyanoborohydride was then passed over the bound peptides for 10 minutes. The reactants were then removed by washing with 10 column volumes of 0.1% formic acid in water, and eluted with 10 volumes of 50% acetonitrile, 0.1% formic acid. The labeled peptides were collected into a low-retention microcentrifuge tube were then taken to dryness in a vacuum centrifuge. The dried peptides were re-constituted in 50 μl of 0.1% TFA and transferred to an autosampler vial for LC-MS/MS analysis.

LC-MS/MS (tandem mass spectrometry) analysis was performed using a Vanquish UHPLC system interfaced with a Lumos Fusion Orbitrap (ThermoFisher) that was operated under the control of Xcalibur 4.1.31.9 software (ThermoFisher). 0.5p g of each peptide digest was loaded onto a 2.1 mm×150 mm C18 CSH Acquity UPLC reversed phase column (1.7 μm particle, Waters Corp.), and separated using a binary gradient as follows: (mobile phase A=0.1% difluoroacetic acid (DFA) in water) 0.5% to 40% of mobile phase B (99.9% acetonitrile, 0.1% DFA) at a flow rate of 200 μl/min and column temperature of 50° C. A top-4 data-dependent acquisition (DDA) MS workflow was used to analyze the LC eluate. Full scan MS spectra were acquired at 120,000 resolution (FWHM) at 200 m/z, and HCD (high energy collisional dissociation) and EThcD (electron transfer dissociation with supplemental HCD energy) MS/MS spectra were acquired in a charge-state dependent manner at 15,000 resolution in the Orbitrap analyzer. The resultant .RAW files from each LC-MS/MS analysis were further processed using Protein Metrics Inc., (PMI) Byonic and Byos software to identify and quantify PTMs. Manual analysis of various spectra was performed using the QualBroswer feature of Xcalibur software.

TABLE 7
MYDGF variants examined by aLCMS or LCMS:

| No. | MYDGF ( . . . ) type | Host system | Expression type |
|---|---|---|---|
| 1 | +S . . . variant | E. coli | Insoluble |
| 2 | −V . . . variant | E. coli | Insoluble |
| 3 | +A . . . variant | E. coli | Insoluble |
| 4 | +G . . . variant | E. coli | Insoluble |
| 5 | wt | CHO | Soluble |

TABLE 8
Intact LCMS MW data for +S MYDGF variant

| | PTM | | Δ MW (Da) | Predicted | Observed | Relative |
|---|---|---|---|---|---|---|
| Sample | category (¹ | Description | predicted | MW (Da) | MW (Da) | percent |
| +S | Full length | unmodified | 0 | 15,920 | 15,920 | 80.9 |
| | N-term | +M | 131 | 16,051 | 16,051 | 3.6 |
| | addition | | | | | |
| | N-term | −SVSE | −402 | 15,518 | 15,518 | 1.5 |
| | truncations | −SVS | −258 | 15,646 | 15,647 | 0.2 |
| | | −SV | −186 | 15,733 | 15,734 | 1.6 |
| | | −S | −87 | 15,832 | 15,833 | 0.6 |
| | Additional | Dehydration | −18 | 15,902 | 15,902 | 1.4 |
| | PTMs | Na+ | 22 | 15,942 | 15,941 | 3.7 |
| | | Carbamoylation | 43 | 15,963 | 15,962 | 4.5 |
| | | gluconoylation | 178 | 16,098 | 16,098 | 2.0 |

(PTMs = Post Translational Modifications). The Na⁺ adduct in intact analysis is a common artefact, not a molecular attribute. The term “N-term” in the peptide map refers to the amino group of the N-terminus. The sequence coverage is 100%. N.D. = PTM < lower limit of detection

TABLE 9
Peptide Mapping LCMS data for +S MYDGF variant

| | | | | Non-binding |
|---|---|---|---|---|
| | | Binding region | | region |
| | | Percent | | Percent |
| Potential modification | | modifica- | | modifica- |
| | Residue | tion | Residue | tion |
| Sequence | n.a. | n.a. | N-term | 84.6* |
| variants | | | unmodified | |
| | | | N-term +M | 3.6* |
| | | | N-term −SVSE | 1.5* |
| | | | N-term −SVS | 0.2* |
| | | | N-term −SV | 1.6* |
| | | | N-term −S | 0.6* |
| Oxidation | Y70, Y72 | N.D. | M34 | 0.5 |
| (16) | | | M49 | 0 |
| | | | M91 | 1.3 |
| | | | W47 | N.D. |
| | | | W65 | N.D. |
| Isomerization | n.a. | n.a. | D11 | N.D. |
| (0) | | | D29 | |
| | | | D56 | |
| | | | D103 | |
| Gluconoylation | K70 | N.D. | N-term | 1.4 |
| (178) | K97 | | K28 | N.D. |
| | K107 | | K77 | N.D. |
| | K126 | | K113 | N.D. |
| | K131 | | K135 | N.D. |
| Carbamoylation | K70 | N.D. | N-term | 3.1 |
| (43) | K97 | | K28 | N.D. |
| | K107 | | K77 | N.D. |
| | K126 | | K113 | N.D. |
| | K131 | | K135 | N.D. |
| Acetylation | K70 | N.D. | N-term | 2.4 |
| (42) | K97 | | K28 | N.D. |
| | K107 | | K77 | N.D. |
| | K126 | | K113 | N.D. |
| | K131 | | K135 | N.D. |
| Dehydration | n.a. | n.a. | D29 | 0 |
| (succinimide) | | | D56 | 4.2 |
| (−18) | | | | |
| Methylation/ | K70 | N.D. | N-term | N.D. |
| glycation/ | K97 | | K28 | N.D. |
| glycosylation | K107 | | K77 | N.D. |
| | K126 | | K113 | N.D. |
| | K131 | | K135 | N.D. |

*determined form intact Mw analysis. The Na⁺ adduct added to original value

TABLE 10
Intact LCMS MW data for −V MYDGF variant

| | PTM | | Δ MW (Da) | Predicted | Observed | Relative |
|---|---|---|---|---|---|---|
| Sample | category | Description | predicted | MW (Da) | MW (Da) | percent |
| −V | Full length | unmodified | 0 | 15,733 | 15,734 | 74.0 |
| | N-term | −SE | −216 | 15,517 | 15,518 | 3.7 |
| | truncations | | | | | |
| | Additional | Dehydration | −18 | 15,715 | 15,716 | 1.9 |
| | PTMs | Na+ | 22 | 15,755 | 15,754 | 4.4 |
| | | Carbamoylation | 43 | 15,776 | 15,776 | 12.1 |
| | | gluconoylation | 178 | 15,911 | 15,912 | 3.9 |

TABLE 11
Peptide Mapping LCMS data for -V MYDGF variant

| | Binding region | | Non-binding region | |
|---|---|---|---|---|
| | | Percent | | Percent |
| Potential | | modifica- | | modifica- |
| modification | Residue | tion | Residue | tion |
| Sequence | n.a. | n.a. | N-term | 78.4* |
| variants | | | unmodified | |
| | | | N-term −SE | 3.7* |
| Oxidation (16) | Y70, Y72 | N.D. | M32 | 2.5 |
| | | | M47 | 3.9 |
| | | | M89 | N.D. |
| | | | W45 | 1.2 |
| | | | W63 | N.D. |
| Isomerization | n.a. | n.a. | D9 | N.D. |
| (0) | | | D27 | |
| | | | D54 | |
| | | | D101 | |
| Gluconoylation | K68 | N.D. | Prot. N-term | 6.7 |
| (178) | K93 | | K28 | N.D. |
| | K105 | | K77 | N.D. |
| | K124 | | K113 | N.D. |
| | K129 | | K135 | N.D. |
| Carbamoylation | K68 | N.D. | Prot. N-term | 9.5 |
| (43) | K93 | | K28 | N.D. |
| | K105 | | K77 | N.D. |
| | K124 | | K113 | N.D. |
| | K129 | | K135 | N.D. |
| Dehydration | n.a. | n.a. | D27 | 3.1 |
| (succinimide) | | | D54 | 0.1 |
| (−18) | | | | |
| Acetylation/ | K68 | N.D. | Prot. N-term | N.D. |
| methylation/ | K93 | | K28 | |
| glycation/ | K105 | | K77 | |
| glycosylation | K124 | | K113 | |
| | K129 | | K135 | |

*determined from intact MW analysis; Na⁺ adduct added to original value

TABLE 12
Intact MW LCMS data for +A MYDGF variant

| | PTM | | Δ MW (Da) | Predicted | Observed | Relative |
|---|---|---|---|---|---|---|
| Sample | category | Description | predicted | MW (Da) | MW (Da) | percent |
| +A | Full length | unmodified | 0 | 15,904 | 15,904 | 83.1 |
| | N-term | −AVSE | −387 | 15,517 | 15,518 | 1.5 |
| | truncations | −AVS | −258 | 15,646 | 15,647 | 0.1 |
| | | −AV | −171 | 15,733 | 15,733 | 0.5 |
| | | −A | −71 | 15,832 | 15,833 | 0.4 |
| | Additional | Dehydration | −18 | 15,886 | 15,886 | 1.7 |
| | PTMs | Na+ | 22 | 15,926 | 15,924 | 5.6 |
| | | Carbamoylation | 43 | 15,947 | 15,946 | 4.6 |
| | | gluconoylation | 178 | 16,082 | 16,082 | 2.5 |

TABLE 13
Peptide Mapping LCMS data for +A MYDGF variant

| | Binding region | | Non-binding region | |
|---|---|---|---|---|
| | | Percent | | Percent |
| Potential | | modifica- | | modifica- |
| modification | Residue | tion | Residue** | tion |
| Sequence | n.a. | n.a. | N-term | 88.7* |
| variants | | | unmodified | |
| | | | N-term −AVSE | 1.5* |
| | | | N-term −AVS | 0.1* |
| | | | N-term −AV | 0.5* |
| | | | N-term −A | 0.4* |
| Oxidation(16) | Y72, | N.D. | M33 | 0.3 |
| | Y74 | | M48 | 0.3 |
| | | | M91 | 1.4 |
| | | | W47 | 1.4 |
| | | | W65 | N.D. |
| Isomerization | n.a. | n.a. | D11 | N.D. |
| (0) | | | D29 | |
| | | | D56 | |
| | | | D103 | |
| Gluconoylation | K70 | N.D. | Prot. N-term | 1.9 |
| (178) | K95 | | K30 | N.D. |
| | K107 | | K79 | N.D. |
| | K126 | | K115 | N.D. |
| | K131 | | K137 | N.D. |
| Carbamoylation | K70 | N.D. | Prot. N-term | 2.1 |
| (43) | K95 | | K30 | N.D. |
| | K107 | | K79 | N.D. |
| | K126 | | K115 | N.D. |
| | K131 | | K137 | N.D. |
| Dehydration | n.a. | n.a. | D29 | 2.8 |
| (succinimide) | | | D56 | 3.3 |
| (−18) | | | | |
| Acetylation/ | K70 | N.D. | Prot. N-term | N.D. |
| methylation/ | K95 | | K30 | |
| glycation/ | K107 | | K79 | |
| glycosylation | K126 | | K115 | |
| | K131 | | K137 | |

*determined form intact mass analysis Na⁺ adduct added to original value
**N-terminal methionine was not observed

TABLE 14
Intact MW LCMS data for +G MYDGF variant

| | PTM | | Δ MW (Da) | Predicted | Observed | Relative |
|---|---|---|---|---|---|---|
| Sample | category | Description | predicted | MW (Da) | MW (Da) | percent |
| +G | Full length | unmodified | 0 | 15,889 | 15,890 | 37.1 |
| | Additional | Dehydration | −18 | 15,871 | 15,872 | 0.8 |
| | PTMs | Na+ | 22 | 15,911 | 15,910 | 2.3 |
| | | Carbamoylation | 43 | 15,932 | 15,932 | 4.2 |
| | N-term | +Met | 131 | 16,021 | 16,021 | 44.1 |
| | addition | Dehydration | −18 | 16,003 | 16,003 | 1.0 |
| | | Na+ | 22 | 16,043 | 16,040 | 3.4 |
| | | Carbamoylation | 43 | 16,064 | 16,067 | 7.1 |

TABLE 15
aLCMS Data (combined Intact MW LCMS and dimethyl-capped peptide level LCMS after trypsination as described*:

| R&D Systems, rMYDGF (human, | | | | | | | | |
|---|---|---|---|---|---|---|---|---|
| wt, untagged) Cat. No. 10231 | | | (+A) MYDGF variant | | | (+S) MYDGF variant | | |
| | | Percent | | | Percent | | | Percent |
| | | N-term | | | N-term | | | N-term |
| | | cleavage | | | cleavage | | | cleavage |
| | | verified | | | verified | | | verified |
| | Whole | by peptide | | Whole | by peptide | | Whole | by peptide |
| | protein | mapping | | protein | mapping | | protein | mapping |
| N-term | analysis | and aLCMS | | analysis | and aLCMS | | analysis | and aLCMS |
| modification | Percent | methodology | | Percent | methodology | | Percent | methodology |
| unmodified | 100.0 | 99.9 | unmodified | 93.4 | 93.2 | unmodified | 93.8 | 93.3 |
| −V | | 0.0 | −A | | 0.2 | −S | | 0.5 |
| −VS | | 0.1 | −AV | | 0.0 | −SV | | 0.0 |
| −VSE | | 0.0 | −AVS | | 0.0 | −SVS | | 0.0 |
| −VSEP | | 0.0 | −AVSE | | 0.0 | −SVSE | | 0.0 |
| Carbamoylation | 0 | | Carbamoylation | 4.3 | | Carbamoylation | 4.2 | |
| Gluconoylation | 0 | | Gluconoylation | 2.3 | | Gluconoylation | 1.9 | |

*N-terminal methionine was not observed in the +A variant; N-terminal methionine was however observed in the +S variant at 2.5%.

### Example 5: Structural Resolution

The +G MYDGF variant (HEK) having a sequence according to SEQ ID NO:3 was manufactured, as described in Polten et al. (2019), and on page 1303, 1′ column and Figure S1, Ebenhoch et al. (2019), page 8 col. 1. The +A MYDGF variant was prepared according to Example 2.

Two-dimensional ¹H/¹⁵N HSQC NMR spectra were collected on a Bruker Avance III 800 MHz spectrometer equipped with a 5 mm z-gradient TCI cryo-probe in 2.5 mm tubes at 310 K. Spectra were recorded using the pulse program hsqcfpf3gpphwg (Bodenhausen & Ruben 1980; Piotto et al. 1992; Sklenar et al. 1993; Mori et al. 1995) from the Bruker catalog with 48 complex points in the indirect dimension, 1024 scans, an interscan delay of 1 s resulting in a total experimental time of 30 h. The hsqcfpf3gpphwg pulse program describes a phase sensitive 2D H-1/X correlation spectrum via a double inept transfer, which uses the f3 channel and employs decoupling during acquisition as well as flip-back pulses and a watergate sequence for water suppression. NMR samples contained 8.5 mg/ml of the respective MYDGF protein in 50 mM sodium phosphate buffer at pH 7.4 containing 50 mM sodium chloride and 9% (v/v) D₂O.

Processing and analysis was performed with Topspin 3.5 (Bruker BioSpin) The ¹H and ¹⁵N chemical shifts of the cross peaks observed in the 2D ¹H/¹⁵N HSQC NMR spectra clearly show that MYDGF is a folded protein for both variants, the +G variant (HEK) and +A variant. The dispersion of cross peaks and their chemical sifts are significantly higher and very different to experimentally determined random chemical shifts (Wishart et al. 1995). A comparison of 2D ¹H/¹⁵N HSQC NMR spectra comparing folded and unfolded proteins are exemplified in this publication (Dyson & Wright, 1995). Differences between random coil and observed chemical shifts are frequently used in NMR structure calculations as restraints for secondary structures (α-helices or β-sheets), (Shen & Bax, 2015).

TABLE 16
Variants examined by NMR

| No. | MYDGF ( . . . ) type | Host system | Expression type |
|---|---|---|---|
| 1 | +G . . . variant (HEK) | HEK | Soluble |
| 3 | +A . . . variant | E. coli | Insoluble |

TABLE 17
1H, 15N chemical shift

| | 1H | 15N |
|---|---|---|
| | chemical shift | chemical shift |
| Peak No. | (ppm) | (ppm) |
| 1 | 10.37 | 129.76 |
| 2 | 10.14 | 129.36 |
| 3 | 9.60 | 129.47 |
| 4 | 9.63 | 128.50 |
| 5 | 9.49 | 128.58 |
| 6 | 9.13 | 129.31 |
| 7 | 8.87 | 129.18 |
| 8 | 9.04 | 128.69 |
| 9 | 9.02 | 128.32 |
| 10 | 9.48 | 126.84 |
| 11 | 9.20 | 126.47 |
| 12 | 8.95 | 126.81 |
| 13 | 8.79 | 127.05 |
| 14 | 8.48 | 127.45 |
| 15 | 8.14 | 128.38 |
| 16 | 7.84 | 129.41 |
| 17 | 8.81 | 125.63 |
| 18 | 8.70 | 125.75 |
| 19 | 8.25 | 126.85 |
| 20 | 9.20 | 125.74 |
| 21 | 9.30 | 124.99 |
| 22 | 9.03 | 124.81 |
| 23 | 8.86 | 124.98 |
| 24 | 8.81 | 124.67 |
| 25 | 8.20 | 125.80 |
| 26 | 7.88 | 125.80 |
| 27 | 8.44 | 124.83 |
| 28 | 8.36 | 124.50 |
| 29 | 7.72 | 124.72 |
| 30 | 7.60 | 123.64 |
| 31 | 7.63 | 122.68 |
| 32 | 7.42 | 122.77 |
| 33 | 7.91 | 122.40 |
| 34 | 8.38 | 122.53 |
| 35 | 8.37 | 123.27 |
| 36 | 8.50 | 123.58 |
| 37 | 8.60 | 123.33 |
| 38 | 8.65 | 123.98 |
| 39 | 8.77 | 123.34 |
| 40 | 8.72 | 122.90 |
| 41 | 8.89 | 122.88 |
| 42 | 9.00 | 121.91 |
| 43 | 8.27 | 121.54 |
| 44 | 9.07 | 122.08 |
| 45 | 9.12 | 121.13 |
| 46 | 9.39 | 121.60 |
| 47 | 9.33 | 120.96 |
| 48 | 9.41 | 120.69 |
| 49 | 9.03 | 120.73 |
| 50 | 8.37 | 120.75 |
| 51 | 8.49 | 120.29 |
| 55 | 9.08 | 119.50 |
| 56 | 8.82 | 119.49 |
| 57 | 8.77 | 119.60 |
| 58 | 8.45 | 119.33 |
| 59 | 7.99 | 119.50 |
| 60 | 8.06 | 120.13 |
| 61 | 8.16 | 120.20 |
| 62 | 8.30 | 119.98 |
| 63 | 8.30 | 119.68 |
| 64 | 8.30 | 118.69 |
| 65 | 7.66 | 121.40 |
| 66 | 7.57 | 121.52 |
| 67 | 7.22 | 121.78 |
| 68 | 7.17 | 121.00 |
| 69 | 7.47 | 119.60 |
| 70 | 7.34 | 119.67 |
| 71 | 7.26 | 119.24 |
| 72 | 7.17 | 119.38 |
| 73 | 7.33 | 118.42 |
| 74 | 6.68 | 120.50 |
| 75 | 6.15 | 117.12 |
| 76 | 7.71 | 117.75 |
| 77 | 8.02 | 117.15 |
| 78 | 8.09 | 117.30 |
| 79 | 8.18 | 117.90 |
| 80 | 8.19 | 117.22 |
| 81 | 8.42 | 118.42 |
| 82 | 8.52 | 117.35 |
| 83 | 8.56 | 116.70 |
| 84 | 8.26 | 116.36 |
| 85 | 8.67 | 117.13 |
| 86 | 8.70 | 116.53 |
| 87 | 8.77 | 117.66 |
| 88 | 8.77 | 116.10 |
| 89 | 8.28 | 115.50 |
| 90 | 8.22 | 115.01 |
| 91 | 7.91 | 115.17 |
| 92 | 7.88 | 115.81 |
| 93 | 7.66 | 115.39 |
| 94 | 7.65 | 113.72 |
| 95 | 8.62 | 114.63 |
| 96 | 8.59 | 113.55 |
| 97 | 9.94 | 115.99 |
| 98 | 9.26 | 118.39 |
| 99 | 9.08 | 118.84 |
| 100 | 8.73 | 116.99 |
| 101 | 7.23 | 115.12 |
| 102 | 6.64 | 115.15 |
| 103 | 6.67 | 113.72 |
| 104 | 7.53 | 112.93 |
| 105 | 6.82 | 112.88 |
| 106 | 6.68 | 112.93 |
| 107 | 7.38 | 112.88 |
| 108 | 7.36 | 112.79 |
| 109 | 7.77 | 111.93 |
| 110 | 7.97 | 112.54 |
| 111 | 8.40 | 112.70 |
| 112 | 8.33 | 111.68 |
| 113 | 8.48 | 111.24 |
| 114 | 7.36 | 111.36 |
| 115 | 6.72 | 111.34 |
| 116 | 7.29 | 110.79 |
| 117 | 6.58 | 110.76 |
| 118 | 6.77 | 109.87 |
| 119 | 7.99 | 108.82 |
| 120 | 8.84 | 110.91 |
| 121 | 8.65 | 109.37 |
| 122 | 8.58 | 109.47 |
| 123 | 8.02 | 107.16 |
| 124 | 7.70 | 106.72 |
| 125 | 7.73 | 102.74 |
| 126 | 10.67 | 128.23 |
| 127 | 9.77 | 127.55 |
| 128 | 9.22 | 130.73 |
| 129 | 6.25 | 123.98 |
| 130 | 10.09 | 132.99 |
| 131 | 9.45 | 131.92 |
| 132 | 9.02 | 132.15 |
| 133 | 9.11 | 123.54 |
| 134 | 9.96 | 122.02 |
| 135 | 6.21 | 109.88 |
| 136 | 6.58 | 111.93 |

The 2D ¹H/¹⁵N HSQC NMR spectra of +G MYDGF variant (HEK), which was found to be active, see Ebenhoch et al. (2019), and +A MYDGF variant are virtually identical. This demonstrates that +A MYDGF has the correct tertiary structure of MYDGF.

### Example 6: Potency Assay in Human Coronary Artery Endothelial Cells

To determine the relative potencies of the [+A] variant relative to the MYDGF+G HEK protein as a reference, a potency assay was performed in human coronary artery endothelial cells (HCAECs). The +A variant was manufactured according to Example 2. Three batches were examined that were designated V301, V302 and V303. The +G variant (SEQ ID NO:3) was produced in HEK cells as described in Polten et al. (2019), page 1303, 1^{st} column and Figure S1, and Ebenhoch et al. (2019), page 8 col. 1, and it was used as an internal activity benchmark.

TABLE 18
Variants examined in the HCAEC assay

| No. | MYDGF ( . . . ) Type | Host system | Expression type |
|---|---|---|---|
| 1 (V301) | +A . . . variant | E. coli | Insoluble |
| 2 (V302) | +A . . . variant | E. coli | Insoluble |
| 3 (V303) | +A . . . variant | E. coli | Insoluble |
| 4 (reference) | +G . . . variant (HEK) | HEK | Soluble |

HCAECs were seeded at a density of 55.000-60.000 cells per well in a 24 well plate in EGM-2 Medium (Lonza) containing 10% fetal calf serum (FCS) in a total volume of 1 ml per well. 24 hours after seeding (cells need to be confluent), the medium of each well was replaced with 1 mL MCDB131-Medium (Life Technologies) containing 2% FCS and incubated for 3-4 hours. After incubation, the cell monolayer was scratched with a pipette tip (200 l) in each well. The tip was used vertically to ensure that the scratch is big enough. The cells were then washed once with MCDB medium containing 2% FCS. Subsequently, 1 mL fresh medium (MCDB containing 2% FCS) was added to each well. The cells were then cultured in the absence (control) or presence of MYDGF protein at different concentrations, wherein each well contained a starting concentration and a serial dilution. Each protein was tested in the following concentrations: 13.3, 19.7, 29.6, 44.4, 66.6, and 100 ng/mL. Human VEGFA (50 ng/mL) served as a positive control. The MYDGF batches V301, V302, V303 were tested in duplicates and head-to-head to the reference. Directly after treatment at T=0 h, a picture was taken from all wells using a microscope (Zeiss Axio Observer Z1 with 50× magnification, 5× objective, with phase contrast setting). The pictures were taken from the middle of the wells, as the optimal contrasts were seen there. The plates were then incubated at 37° C. After an incubation time of 16 hours (T=16 h), pictures of each well were taken again as described above.

For determination of the activity, the recovery in the assay was calculated by measuring the cell free area using axiovision software or ImageJ in pictures at 0 h and in pictures at 16 h, respectively. Recovery (%) was calculated as [(cell free area at 0 h−cell free area at 16 h)/cell free area at 0 h]×100. Two experiments were summarized to perform 4 parameter logistic (4-PL) curve fits and calculate EC₅₀ values (GraphPad Prism software, version 9.1.0). The EC₅₀ values from the different batches and the Reference were applied to calculate the relative potency compared to the reference: Potency [%]=EC₅₀ of [+G]-HEK)/EC₅₀ of test batch)×100.

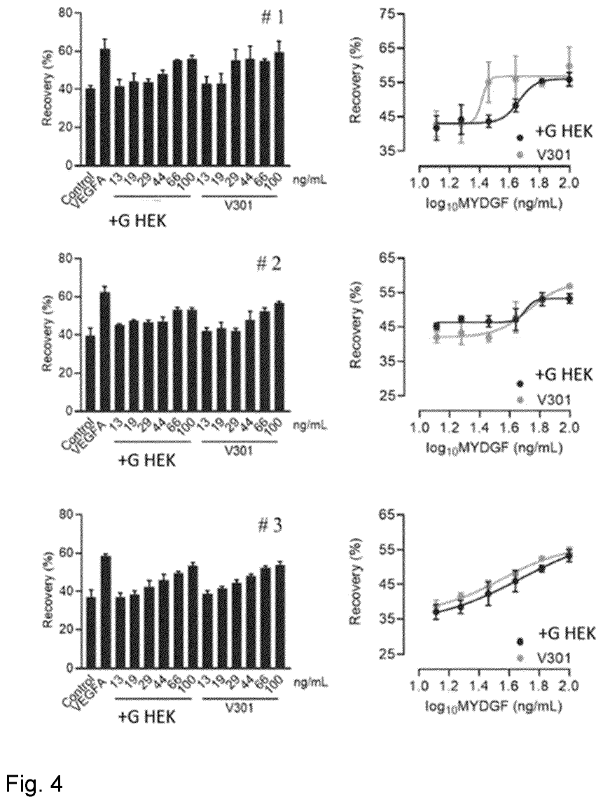

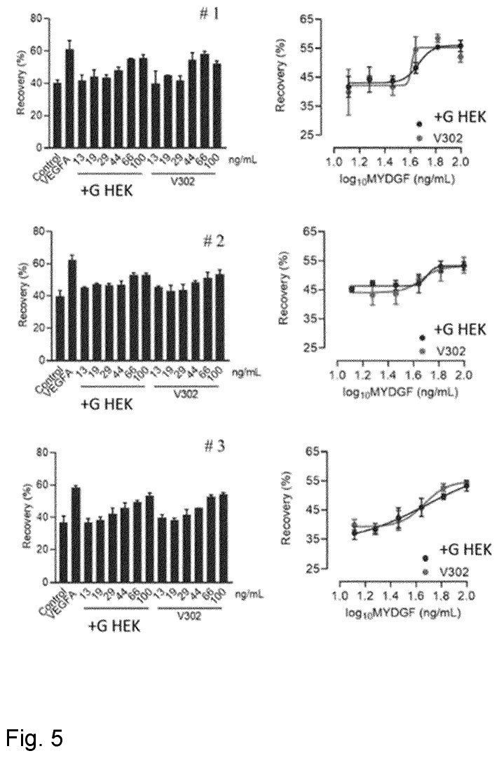

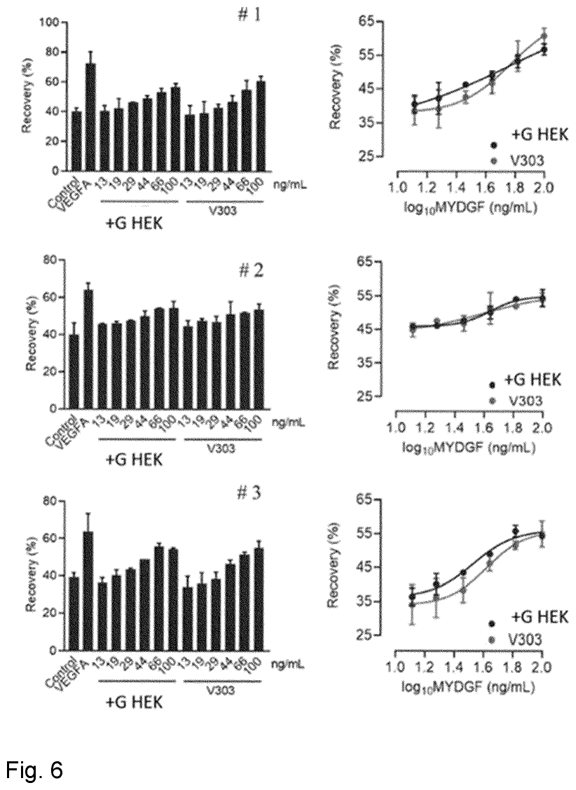

TABLE 19
EC₅₀ and potency values of different batches of the [+A]
variant determined in the HCAEC assay

| | Sample | EC₅₀ (ng/mL) | Potency (%) |
|---|---|---|---|
| FIG. 4 | | | |
| #1 | +G HEK | 46.2 | 100 |
| | V301 | 26.1 | 177 |
| #2 | +G HEK | 50.9 | 100 |
| | V301 | 53.7 | 95 |
| #3 | +G HEK | 47.9 | 100 |
| | V301 | 35.2 | 136 |
| FIG. 5 | | | |
| #1 | +G HEK | 46.2 | 100 |
| | V302 | 40.6 | 114 |
| #2 | +G HEK | 50.9 | 100 |
| | V302 | 46.2 | 110 |
| #3 | +G HEK | 47.9 | 100 |
| | V302 | 46.3 | 103 |
| FIG. 6 | | | |
| #1 | +G HEK | 58.1 | 100 |
| | V303 | 58.5 | 99 |
| #2 | +G HEK | 43.8 | 100 |
| | V303 | 39.5 | 111 |
| #3 | +G HEK | 33.9 | 100 |
| | V303 | 41.9 | 81 |

Results: As can be seen from Table 18, the three +A MYDGF variant production batches (V301, V302, V303) all show biological activities comparable to the reference. In HCAECs, the following relative potencies compared to the reference (set to 100%) were determined: V301 (177, 95, and 136%), V302 (114, 110, and 103%), V303 (99, 111, and 81%). Pooled cell migration experiments resulted in the EC₅₀ values of 37.4, 42.1 and 47.8 ng/mL for the batches V301, V302 and V303, respectively. When pooling the data from all batches (V301 & V302, V303) and conducting a curve fit, an EC₅₀ value of 41.1 ng/mL was calculated. The EC₅₀ value for the corresponding reference calculated in the same way was 43.0 ng/mL.

### Example 7: Potency Assay in Neonatal Rat Cardiomyocytes

Neonatal rat cardiomyocytes (NRCM) were seeded in 96 well plates and were subjected to simulated ischemia/reperfusion (I/R) in the absence (control) or presence of different MYDGF batches. I/R was simulated as described earlier by Korf-Klingebiel et al., (2015). The reference protein was assayed in head-to-head comparisons. Each protein was tested in the following six concentrations: 13.3, 19.7, 29.6, 44.4, 66.6, and 100 ng/mL. Mouse IGF-1 (50 ng/mL) served as a positive control. Metabolite activity was assessed by the MTS assay (Promega). The MYDGF batches V301, V302, and V303 were tested in 3-4 technical replicates in 2-3 experiments. Experiments were summarized to perform 4-PL curve fits and calculate EC₅₀ values. The EC₅₀ values from the different batches and the reference were applied to calculate the relative potency relative to the reference (see above).

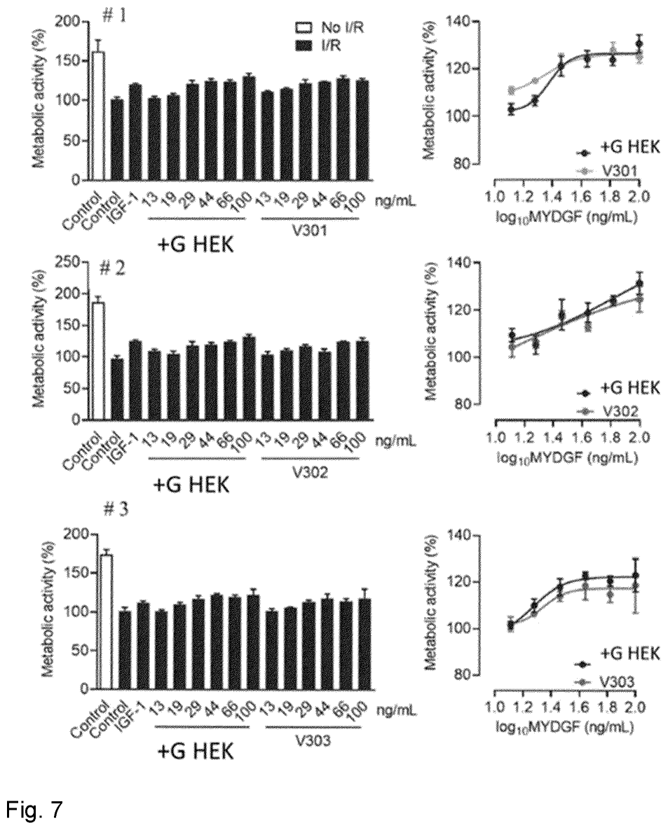

TABLE 20
Potency values of different batches of the [+A] variant
determined in the neonatal rat cardiomyocyte assay

| FIG. 7 | Sample | EC₅₀ (ng/mL) | Potency |
|---|---|---|---|
| #1 | +G HEK | 24.0 | 100 |
| | V301 | 22.1 | 108 |
| #2 | +G HEK | 65.6 | 100 |
| | V302 | 15.2 | 431 |
| #3 | +G HEK | 18.6 | 100 |
| | V303 | 21.9 | 85 |

Results: In neonatal rat cardiomyocytes, the following relative potencies compared to the reference (set to 100%) were determined: V301 (108%), V302 (431%), V303 (85%). The potency values for MYDGF batches V301 and V303 indicated a biological activity comparable to the reference. Although the metabolic activity for batch V302 was similar to the reference (bar graphs in FIG. 7) the non-ideal curve fit resulted in a 4-fold higher potency. When pooling the data from all batches (V301 & V302 & V302) and conducting a curve fit, an EC₅₀ value of 22.2 ng/mL was calculated. The EC₅₀ value for the corresponding reference calculated in the same way was 24.5 ng/mL.

### Example 8: Mouse Myocardial Infarction Assay

To compare the efficacies of human and murine MYDGF treatments on myocardial infarction (MI) healing in mice, a mouse model of myocardial infarction was used. FVB/N mice were subjected to sham or verum (ischemia/reperfusion) surgery, treated with human or murine recombinant MYDGF, and followed-up for 28 days. The human and murine MYDGF proteins used in the assay are depicted in Table 20.

TABLE 21
Variants examined in the mouse myocardial infarction assay.

| No. | MYDGF ( . . . ) Type | Host system | Expression type |
|---|---|---|---|
| 1 | Murine Mydgf-His | HEK | Soluble |
| | variant | | |
| 2 | +G . . . variant (HEK) | HEK | Soluble |

The +G MYDGF variant (HEK) having a sequence according to SEQ ID NO:3 was manufactured, essentially as described in Polten et al. (2019), and on page 1303, 1′ column and Figure S1, Ebenhoch et al. (2019), page 8 col. 1. The murine MYDGF-His variant was prepared according to Korf-Klingebiel et al. (2021), Suppl. Material.

Heart failure-prone FVB/N mice were subjected to sham (thoracotomy without ischemia/reperfusion; No I/R) or I/R (ischemia/reperfusion) surgery. Mice were treated with human or murine MYDGF (10 μg bolus+10 g/d pump for 7 days, Model 1007D) or diluent only (placebo). Serial echocardiographies were performed (on days 6 and 28) and mice were followed-up for 28 days. At the end of experiment, hearts were collected and scar size was determined by Masson's trichrome staining and capillary density by fluorescent IB4/WGA staining. Statistical significance was assessed by one-way ANOVA with Dunnett's multiple comparison post hoc test. *P<0.05, **P<0.01, ***P<0.001 vs. sham (vs. all VR groups for FAC). ##P<0.01, ###P<0.001 vs. placebo.

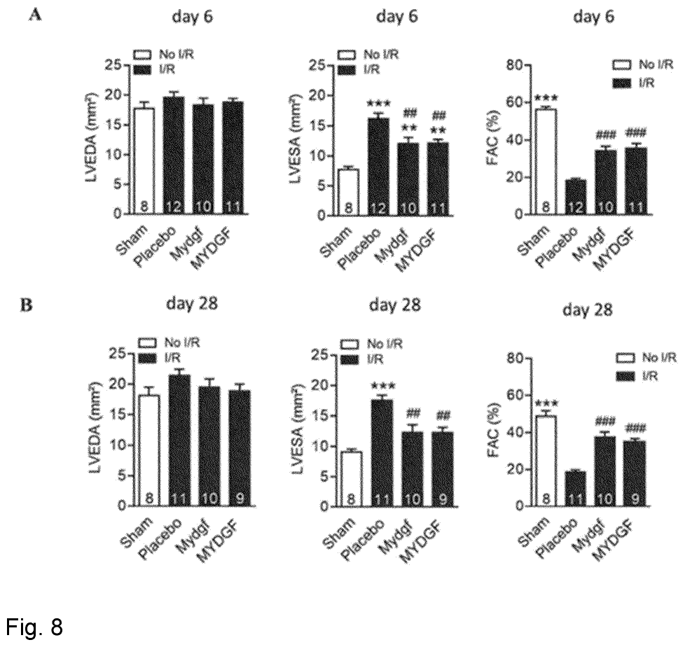

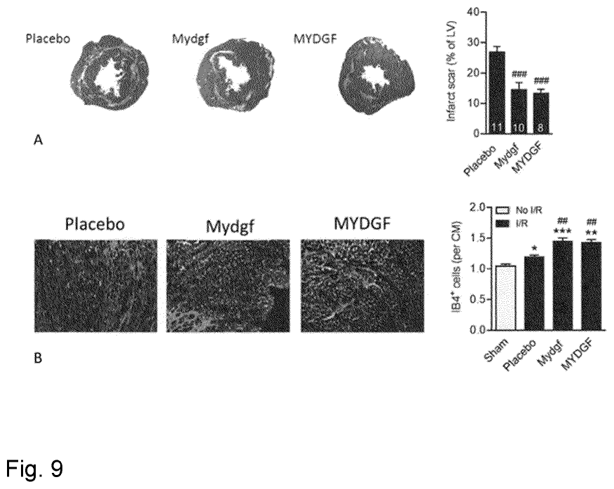

Results: It was found that protein therapy with human MYDGF (“MYDGF”) improved cardiac function (fractional area change, FAC) over placebo treatment by 17.5% on day 6 and 16.7% on day 28. Murine MYDGF (“Mydgf”) increased FAC over placebo treatment by 16.1% on day 6 and 19.0% on day 28 (see FIG. 8). As can be seen in FIG. 9, infarct scar was reduced by both recombinant MYDGF proteins (16.5% with human MYDGF; 12.4% with murine MYDGF vs. placebo). Further, MYDGF protein therapy increased capillary density in the infarct border zone after MI by 21.8% over placebo treatment for the human protein and by 19.1% over placebo treatment for the murine protein (see FIG. 9). Thus, both treatments significantly improved MI healing as assessed by cardiac functional improvement, reduced scar size, and increased capillary density in the infarct border zone.

### LITERATURE

1. Botnov, V. et al. (2018), J Biol Chem, 293(34), 13166-13175.
2. Ebenhoch, R. et al. (2019), Nat Commun 10, 5379.
3. Korf-Klingebiel, M. et al. (2015), Nat Med 21(2): 140-9.
4. Korf-Klingebiel, M. et al. (2021), Circulation. 144(15): 1227-40.
5. Polten, F. et al. (2019), Anal Chem, 91, 1302-1308.
6. Tolonen, A. C. et al. (2011), Mol Systems Biol 7(1).
7. Zhao, L. et al. (2020), J Cell Mol Med, 24 (2): 1189-1199.
8. Frottin, F. et al. (2006), The Proteomics of N-terminal Methionine Cleavage, Molecular & Cellular Proteomics, Volume 5, Issue 12, 2336-2349.
9. Bodenhausen G. & Ruben D. J. (1980), Chem. Phys. Lett. 69, 185.
10. Piotto, M. et al. (1992), J Biomol NMR, 2, 661-666.
11. Sklenar, V. et al. (1993), J Magn Reson, Series A 102, 241-245.
12. Mori, S. et al. (1995), J Magn Reson B 108, 94-98.
13. Wishart, D. S. et al. (1995), J Biomol NMR, 5, 67-81.
14. Dyson, H. J. & Wright, P. E. (1998), Nat Struct Biol, 5, 499-503.
15. Shen Y. & Bax A. (2015), Methods Mol Biol, 1260:17-32.
16. CN 111544572 A
17. EP2918676B1
18. U.S. Pat. No. 5,744,328 B
19. U.S. Pat. No. 7,851,433 B
20. US 2015/0291683 A1
21. WO 2011/154349 A2
22. Peternel, S. & Komel, R. (2010), Microbial Cell Factories, 9:66.
23. Eggenreich, B. et al. (2020), Journal of Biotechnology 324S, 100022.
24. Brinson, R. G. et al. (2019), mAbs, 11, 94-105.
25. WO 2021/148411
26. WO 2014111458

1. Method for the recombinant expression of a MYDGF protein in a bacterial host cell, comprising
(a) providing a host cell that comprises a nucleic acid encoding a protein which after maturation consists of 143 amino acids having the amino acid sequence of SEQ ID NO:1 or SEQ ID NO:2;
(b) culturing the host cell under conditions that allow the expression of the protein;
(c) isolating inclusion bodies containing MYDGF protein from the host cell; and
(d) solubilising the inclusion bodies and refolding the MYDGF protein.

2. Method of claim 1, wherein step (a) comprises
(i) providing a host cell that comprises a nucleic acid that contains an open reading frame, flanked by start and stop codon, according to the sequence of SEQ ID NO:11 or SEQ ID NO:12, and preferably according to the sequence of SEQ ID NO:11, operably linked to a promotor; or
(ii) providing a host cell that comprises a nucleic acid encoding a protein which before maturation consists of 144 amino acids having the amino acid sequence of SEQ ID NO:15 or SEQ ID NO:16, and preferably the amino acid sequence of SEQ ID NO:15.

3. Method of claim 1, wherein step (a) comprises providing a host cell that comprises a nucleic acid of SEQ ID NO:7 or SEQ ID NO:8, and preferably a nucleic acid of SEQ ID NO:7.

4. Method of claim 1, wherein said maturation is the removal of the N-terminal methionine residue.

5. Method of claim 4, wherein said removal of the N-terminal methionine residue is effected by one or more host cell-derived aminopeptidases.

6. Method of claim 1, further comprising (e) obtaining after step (d) a refolded MYDGF protein of 143 amino acids having the amino acid sequence of SEQ ID NO:1 or SEQ ID NO:2, and preferably the amino acid sequence of SEQ ID NO:1.

7. Method of claim 1, wherein refolding of the protein in step (d) comprises the incubation of the protein in the presence of urea.

8. Method of claim 1, wherein said method further comprises (f) purifying the MYDGF protein.

9. Method of claim 8, wherein step (f) comprises ultrafiltration, diafiltration, hydrophobic interaction chromatography and/or anion ion exchange chromatography.

10. Method of claim 9, wherein anion exchange chromatography or hydrophobic interaction chromatography step is performed by contacting the MYDGF protein to the chromatography resin material under conditions that allow for the adsorption of the MYDGF protein to the resin, optionally washing the resin, and eluting the MYDGF protein from the resin.

11. Method of claim 10, wherein the adsorption of the MYDGF protein to the anion exchange chromatography resin is performed under conditions of low ionic strength.

12. Method of claim 11, wherein adsorption is performed at a conductivity of less than 3 mS/cm, less than 2 mS/cm, less than 1.5 mS/cm or less than 1 mS/cm.

13. Method of claim 9, wherein desorption of the MYDGF protein from the anion exchange resin is effected by increasing the salt concentration and/or lowering the pH of the liquid phase.

14. Method of claim 13, wherein the bacterial host cell is an Escherichia coli cell.

15. Method of claim 14, wherein the Escherichia coli cell is an Escherichia coli cell of strain BL21 or a derivative strain thereof.

16. Method of claim 1, wherein said nucleic acid is DNA or RNA.

17. Method of claim 16, wherein said nucleic acid comprises the sequences of SEQ ID NO:11 or SEQ ID NO:12.

18. Method of claim 1, wherein the nucleic acid is contained in a vector.

19. Method of claim 18, wherein said vector is a prokaryotic expression vector.

20. Method of claim 18, wherein said vector comprises a T7 promoter.

21. Method of claim 18, wherein said vector comprises or consists of the sequences of SEQ ID NO:7 or SEQ ID NO:8.

22. Composition obtainable from the method of claim 1, wherein said composition comprises a protein of 143 amino acids having the amino acid sequence of SEQ ID NO:1 or SEQ ID NO:2.

23. Composition of claim 22, wherein said composition comprises less than 1% (w/w) of protein molecules that are shorter than 143 amino acids, as measured by liquid chromatography mass spectrometry (LCMS).

24. Composition of claim 22, wherein said composition comprises less than 20 pg/mg, preferably less than 15 pg/mg, more preferably less than 10 pg/mg, and most preferably less than 5 pg/mg, less than 3 pg/mg, less than 2 pg/mg or less than 1 pg/mg host cell DNA.

25. Composition of claim 22, wherein said composition comprises less than 0.2 EU/mg, and preferably less than 0.1 EU/mg or 0.08 EU/mg bacterial endotoxin.

26. Composition of claim 22, wherein less than 8% (w/w), and preferably less than 7% (w/w), less than 6% (w/w) or less than 5% (w/w), less than 4% (w/w), less than 3% (w/w), or less than 2% (w/w) of the proteins in said composition are carbamoylated.

27. Composition of claim 22, wherein less than 6% (w/w), and preferably less than 5% (w/w), less than 4% (w/w) or less than 3% (w/w), or less than 2% (w/w) of the proteins in said composition are gluconoylated.

28. Composition of claim 22, wherein less than 8%, and preferably less than 7%, less than 6% or less than 5% of the MYGDF proteins in the composition of the invention are carbamoylated, wherein the percentage is based on the sum of the peak intensities of unmodified MYDGF protein as well as annotated post-translational modification (PTM) species of MYDGF in a deconvoluted intact mass spectrum of the MYDGF protein in the composition.

29. Composition of claim 22, wherein less than 6%, and preferably less than 5%, less than 4% or less than 3% of the MYDGF proteins in the composition of the invention are gluconylated, wherein the percentage is based on the sum of the peak intensities of unmodified MYDGF protein as well as annotated post-translational modification (PTM) species of MYDGF in a deconvoluted intact mass spectrum of the MYDGF protein in the composition.

30. Composition of claim 22, wherein said composition comprises urea.

31. Composition of claim 22, wherein said composition comprises a protein of 143 amino acids having the amino acid sequence of SEQ ID NO:1 and the ratio of the signal for the protein according to SEQ ID NO:1 and the signals for shorter variants in liquid chromatography mass spectrometry (LCMS) after reductive dimethylation (stable isotope dimethyl labelling, SIDL) is at least 50, and preferably more than 100, 200, 300 or 400, wherein only signals from non-carbamoylated and non-gluconoylated proteins are used for calculating said ratio.

32. Composition of claim 22, wherein said composition comprises a protein of 143 amino acids having the amino acid sequence of SEQ ID NO:2 and the ratio of the signal for the protein according to SEQ ID NO:2 and the signals for shorter variants in liquid chromatography mass spectrometry (LCMS) after reductive dimethylation (stable isotope dimethyl labelling, SIDL) is at least 50, and preferably more than 75, 100, 150 or 175, wherein only signals from non-carbamoylated and non-gluconoylated proteins are used for calculating said ratio.

33. Composition of claim 22, wherein said composition comprises a protein which is folded such that more than 70%, and preferably more than 80%, more than 90%, or more than 95%, of the ¹H and/or ¹⁵N peaks in the two-dimensional nuclear magnetic resonance spectroscopy (2D-NMR) map result in combined chemical shift deviation (CCSD) values below 0.01 ppm when compared to the corresponding peaks in Table 1.

34. Composition of claim 22, wherein said composition comprises the MYDGF protein with a monomer content of more than 95%, 96%, 97%, 98% or 99%.

35. Use of a composition of claim 22 for the preparation of a pharmaceutical composition.

36. Pharmaceutical composition comprising a composition of claim 22.

37. Pharmaceutical composition of claim 36, further comprising a pharmaceutically acceptable carrier.

38. Pharmaceutical composition of claim 36, wherein said composition is formulated for parental administration.

39. Pharmaceutical composition of claim 38, wherein said composition is formulated for intravenous, intraarterial or intracoronary administration.

40. Pharmaceutical composition of claim 39, wherein said composition is formulated for intravenous administration.

41. Composition of claim 22 for use as a medicament.

42. A method comprising administering a composition according to claim 22 to a subject in need thereof, wherein the method is for
(i) treating or preventing a disease or condition selected from the group consisting of injury, wounding, ischemia, reperfusion injury, trauma, mechanical overload, intoxication, surgery, primary or acquired cardiomyopathy, postischemic contractile dysfunction, myocardial infarction, preferably acute myocardial infarction, angina pectoris, heart failure, inflammation of the heart, heart insufficiency, hypertrophy, and fibrosis;
(ii) promoting or improving heart tissue regeneration, cardiomyocyte proliferation, neovascularisation, heart function or left ventricular systolic function after myocardial infarction;
(iii) protecting cardiomyocyte from death, e.g. through apoptosis or necrosis; or
(iv) decreasing infarct size after myocardial infarction, preferably acute myocardial infarction.

43. The method of claim 42, wherein said cardiomyopathy is inherited cardiomyopathy or cardiomyopathy caused by spontaneous mutations.

44. The method of claim 42, wherein said cardiomyopathy is acquired cardiomyopathy, preferably ischemic cardiomyopathy caused by atherosclerotic or other coronary artery diseases, cardiomyopathy caused by infection or intoxication of the myocardium, hypertensive heart disease caused by pulmonary arterial hypertension and/or arterial hypertension and diseases of the heart valves.

45. The method of claim 42, wherein said cardiomyopathy is selected from the group consisting of hypertrophic cardiomyopathy (HCM or HOCM), arrythmogenic right ventricular cardiomyopathy (ARVC), isolated ventricular non-compaction mitochondrial myopathy, dilated cardiomyopathy (DCM), restrictive cardiomyopathy (RCM), Takotsubo cardiomyopathy, Loeffler endocarditis, diabetic cardiomyopathy, alcoholic cardiomyopathy, or obesity-associated cardiomyopathy.

46. The method of claim 42, wherein said heart failure is chronic heart failure.

47. The method of claim 46, wherein said heart failure or chronic heart failure is heart failure with preserved ejection fraction (HFpEF), heart failure with reduced ejection fraction (HFrEF), or heart failure with mid-range ejection fraction (HFmrEF).

48.-50. (canceled)
